(19) United States
(12) Patent Application Publication
(10) Pub. No.: US 2022/0127678 A1
(43) Pub. Date: Apr. 28, 2022

(54) BIOMARKER PANEL FOR DIAGNOSIS AND PROGNOSIS OF CANCER

(71) Applicant: UNIVERSITÄT HEIDELBERG, Heidelberg (DE)

(72) Inventors: Sarah SCHOTT, Heidelberg (DE); Christof SOHN, Heidelberg (DE); Aoife GAHLAWAT, Heidelberg (DE); Tania WITTE TOBAR, Leimen (DE)

(21) Appl. No.: 17/429,997

(22) PCT Filed: Feb. 21, 2020
(86) PCT No.: PCT/EP2020/054667

§ 371 Date: Aug. 11, 2021

(30) Foreign Application Priority Data
Feb. 21, 2019 (EP) 19158682.5
May 14, 2019 (EP) 19174483.8

(51) Int. Cl. C12Q 1/6886

(52) U.S. Cl. CPC C12Q 1/6886; C12Q 2600/112; C12Q 2600/178; C12Q 2600/154; C12Q 2600/158; C12Q 2600/118

(57) ABSTRACT

The present invention relates to a method for diagnosing or prognosing cancer comprising determining in vitro cytosine methylation levels within marker genes and/or determining expression levels of miRNA markers.

### CROSS REFERENCE TO RELATED APPLICATION(S)

This application is a U.S. National Phase of International Application No. PCT/EP2020/054667, filed on Feb. 21, 2020, which claims priority to European Patent Application No. EP 19158682.5, filed Feb. 21, 2019 and European Patent Application No. EP 19174483.8, filed May 14, 2019, all of which are incorporated by reference herein in their entirety.

The present invention relates to panels of methylation and miRNA markers as well as their use in the prognosing, diagnosing and/or treatment of cancer, means for detecting said marker and kits comprising said means.

### BACKGROUND OF THE INVENTION

Cancer is one of the most important medical and health problems in the world. As the leading cause of death worldwide, there were 12.4 million new cancer cases and 7.6 million cancer related deaths in 2008. It has been predicted that the deaths from cancer worldwide is continuously rising and 12 million deaths would be caused by cancer in the year of 2030. Breast cancer is the most common cancer among women. About one out of nine women will develop breast cancer during her life (Feuer, E. J., et al., The lifetime risk of developing breast cancer; J Natl Cancer lnst 85, 892-897 (1993)). Worldwide approximately 1.3 million women develop breast cancer each year. Mortality rates have continued to decrease over the years due to all the efforts and advances made in early diagnosis and treatment (Jemal A, Bray F, Center M M, Ferlay J, Ward E, Forman D. Global cancer statistics. CA Cancer J Clin 2011; 61:69-90). Nevertheless, thousands of women die from this disease each year. In US women the overall five-year survival is 98% when diagnosed at an early stage as opposed to 23% when the disease has already spread to distant organs. Thus, early breast cancer detection belongs to one of the major challenges in the struggle against this disease. Mammographic screening is currently applied as the diagnostic standard. However, it has limitations due to its use of ionizing radiation and a false positive rate of 8-10%, also depending on the age of the individuals to be screened (Taplin S et al.; J Natl Cancer Inst 2008; 100: 876-87).

Most of the breast cancers occur sporadic, whereas familial breast cancer accounts for about 10% of all breast cancer cases (Fackenthal et al.; Nat Rev Cancer 7, 937-948 (2007)). Mutations in the main breast cancer related genes, BRCA1 and BRCA2 account for 25% and other intermediate- and low-penetrance genes for about 5% of all familial cases (Yang, R. & Burwinkel, B. (eds.); Familial risk in breast cancer, 251-256 (Springer, 2010)). Recent genome-wide association studies (GWAS) and single candidate gene approaches have been quite successful in detecting genetic low-risk variants for breast cancer (Thomas, G., et al., Nat Genet 41, 579-584 (2009); Cox, A., et al., Nat Genet 39, 352-358 (2007); Stacey, S. N., et al., Nat Genet 40, 703-706 (2008); Ahmed, S., et al., Nat Genet 41,585-590 (2009); Easton, D. F., et al., Nature 447, 1087-1093 (2007); Milne, R. L., et al., J Natl Cancer Inst 101, 1012-1018 (2009); Frank, B., et al., J Natl Cancer Inst 100,437-442 (2008)). However, a large number of breast cancer risk factors remain to be explored.

Compared to BC, ovarian cancer (OvCa) is comparable rare in occurrence, but is the leading cause of death from gynaecologic cancers because of its high malignancy. In 2008, 225,000 women were diagnosed with ovarian cancer worldwide, and 140,000 of these women died from the disease. Typically, women with the OvCa present with few early symptoms, and thus nearly three-quarters of ovarian cancer cases present at an advanced stage, with the disease spread well beyond the ovaries. Pancreatic cancer (PaCa) is the most aggressive of all epithelial malignancies. With 279,000 new diagnoses of PaCa worldwide, the 5-year overall survival rate of PaCa patients is less than 5%. Although recent genome-wide association studies (GWAS) have successfully detected several genetic variants associated with the risk of BC, OvCa and PaCa, no valuable marker for the early detection of BC has been identified.

Metastatic breast cancer (MBC) is a major health issue, worldwide. Current treatment strategies target primarily palliative care with very few cases being cured. An alternate approach of tackling MBC is development of screening methods and applying biomarkers to identify high risk groups and therapy response. This could facilitate decision making for clinicians and help them adopt the appropriate treatment regime for the patients.

Circulating tumor cells (CTC) have been proposed as an FDA approved independent prognostic marker for metastasis, specifically for progression-free survival and overall survival. A cardinal cut off of greater than 5 CTCs per 7.5 ml of blood has been defined as CTC positive (Cristofanilli et al., N Engl J Med. 2004 Aug. 19; 351(8):781-91). However, it is important to note that a significant fraction of patients with overt distant metastases are negative for CTCs. This could be partly contributed to the phenomenon of epithelial-mesenchymal transition in CTCs, in which case they can be missed by enumeration techniques that exploit the expression of epithelial markers such as EpCAM or cytokeratin-8, -18 and -19.

Beside CTCs, also protein based circulating tumor markers like carcinoembryonic antigen (CEA) and carbohydrate antigen 15-3 (CA 15-3) are widely used as prognostic markers, as well as in monitoring breast cancer treatment success and follow-up (Uehara et al., Int J Clin Oncol 2008; 13:447-51; Harris et al., J Clin Oncol 2007; 25:5287-312). However, the sensitivity of these markers is low. Therefore, new sensitive and specific as well as minimally invasive markers are needed.

Epigenetic changes are defined as changes in gene expression that are not due to any alterations in the genomic DNA sequence. Aberrant epigenetic signatures have been considered as a hallmark of human cancer (Esteller, M. Nat Rev Genet 8, 286-298 (2007)). One of the most important epigenetic signatures, DNA methylation, has critical roles in the control of gene activities and in the architecture of the nucleus of the cell (Weber, M., et al., Nat Genet 37, 853-862 (2005)). Furthermore, unlike genetic markers or variants, DNA methylation is principally reversible. Therefore, the methylation profile of specific genes is considered as therapeutic target (Mack, G. S. J Natl Cancer Inst 98, 1443-1444 (2006)). Meanwhile, due to the variable character, DNA methylation may serve as a link between environmental factors and the genome. DNA methylation modulated by environmental factors or aging may alter the expression of critical genes of cells and consequently induce malignant transformation of cells or even a cancer (Widschwendter, M., et al., PLoS One 3, e2656 (2008)).

As an early event in the development of cancer, changes of DNA methylation are particularly promising as markers for the early detection of cancer. Recent studies have shown that methylation analysis of blood cell DNA can serve as a reliable and robust marker. Intensive studies have disclosed altered DNA methylation signatures in cancer on the somatic level, whereas only a few studies with candidate-gene-approach have analysed methylation signatures in peripheral blood DNA in cancer.

Previous studies have explored hypermethylation in the promoter regions of tumor suppressor genes and hypomethylation in the promoter regions of oncogenes in breast cancer compared to their normal adjacent tissues (Ito, Y., et al. Hum Mol Genet 17, 2633-2643 (2008); Potapova et al., Cancer Res 68, 998-1002 (2008); Radpour et al., Oncogene 28, 2969-2978 (2009); Widschwendter, M. & Jones, P. A. Oncogene 21, 5462-5482 (2002)). Very few studies have focused on the methylation signatures in the peripheral blood DNA and-breast cancer risk. In these studies, only specific genes, like BRCA1 (Iwamoto et al., Breast Cancer Res Treat 129, 69-77 (2011)), ATM (Flanagan, et al., Hum Mol Genet 18, 1332-1342 (2009)), and genes in specific pathways (Widschwendter et al. (2008), loc. cit.) have been investigated.

There is thus a need in the art for the identification of further markers of breast cancer, ovarian cancer and other cancers, preferably allowing the identification of afflicted subjects by obtaining a sample by a means of low invasiveness, e.g. by taking a blood sample.

There is thus an urgent need in the art for improved methods for the diagnosis and prognosis of cancer, in particular breast cancer, ovarian cancer and pancreatic cancer. These methods would preferably be also used in preventive screening of apparently healthy subjects; a low grade of invasiveness would be preferred.

### SUMMARY OF THE INVENTION

In a first aspect, the present invention provides a method of diagnosing or prognosing cancer in a subject, comprising the steps of determining in vitro in a sample obtained from said subject
a) the cytosine methylation of at least one CpG dinucleotide within at least one gene selected from the group consisting of HYAL2, MGRN1, RPTOR, SLC22A18, FUT7, RAPSN and S100P and/or
b) the expression level of at least one miRNA selected from the group consisting of miR-148b, miR-409, miR-652, miR-200c, miR-375, miR-320b and miR-141 with the proviso that the at least one miRNA comprises at least one miRNA selected from the group consisting of miR-200c-3p, miR-375 and miR-320b,
   wherein the method optionally further comprises determining the expression level of miR-451a, wherein a decreased level of cytosine methylation of at least one CpG dinucleotide within the at least one gene and an altered expression level of the at least one miRNA is indicative of the present and/or future cancer disease state in said subject.

In a second aspect the present invention provides a method for diagnosing cancer or for screening for cancer, comprising predicting or detecting the cancer according to the first aspect of the invention.

In a third aspect the present invention provides a method for monitoring a subject having an increased risk of developing cancer, comprising predicting or detecting repeatedly the cancer according to the first aspect of the invention.

In a fourth aspect the present invention provides a method for monitoring cancer treatment of a subject, comprising predicting or detecting the cancer according to the first aspect of the invention repeatedly across the treatment period.

In a fifth aspect the present invention provides a method for assessing the response of a subject to a cancer treatment, comprising predicting or detecting the cancer according to the first aspect of the invention during and/or after the treatment.

In a sixth aspect the present invention provides a method for treating a subject having cancer detected according to the method according to the first aspect of the invention, further comprising administering a cancer therapy to the subject.

In a seventh aspect the present invention provides a kit comprising oligonucleotides for specifically detecting:
the level of cytosine methylation of at least one CpG dinucleotide within and/or expression level at least one gene selected from the group consisting of HYAL2, MGRN1, RPTOR, SLC22A18, FUT7, RAPSN and S100P, and/or
the expression level of at least one miRNA selected from the group consisting of 148b, miR-409-3p, miR-652-3p, miR-200c-3p, miR-375, miR-320b, miR-451a and miR-141 with the proviso that the at least one miRNA comprises at least one miRNA selected from the group consisting of miR-200c-3p, miR-375, miR-320b and miR-451a.
   In an eight aspect the present invention provides the use of the kit of the seventh aspect of the invention for predicting, prognosing and/or diagnosing cancer, preferably breast cancer, ovarian cancer, and/or pancreatic cancer, preferably breast cancer and ovarian cancer.

### LIST OF FIGURES

In the following, the content of the figures comprised in this specification is described. In this context please also refer to the detailed description of the invention above and/or below.

FIG. 1: refers to a list of 15 markers used. The list of genes provided are those genes of which cytosine methylation in CpG dinucleotides was determined. The right column indicates the preferred CpG measured, preferably for breast cancer early diagnosis. Furthermore, are preferred miRNAs listed, whose expression level has been determined. Furthermore, the preferred clinical marker—age—and a measure of the total quantity of circulating miRNAs—Qubit—is listed.

FIG. 2: refers to six different models tested in the HEIScreen cohort consisting of ovarian cancer patients. The 49 markers tested in model 1 are: Age of patient, CA125, Qubit, miR-409, miR-652, miR-148b, miR-375, miR-200c, miR-320b, RPTOR_CpG1, RPTOR_CpG2, RPTOR_CpG5, RPTOR_CpG6, RPTOR_CpG8, FUT7_CpG1, FUT7_CpG2, FUT7_CpG3, FUT7_CpG4, FUT7_CpG6, FUT7_CpG7, S100P_CpG2.3, S100P_CpG4, S100P_CpG7, S100P_CpG8, S100P_CpG9, S100P_CpG10.11.12, SLC22A18_CpG1, SLC22A18_CpG3, SLC22A18_CpG4, SLC22A18_CpG6, SLC22A18_CpG8, HYAL2_CpG1, HYAL2_CpG2, HYAL2_CpG3, HYAL2_CpG4, MGRN1_CpG4, MGRN1_CpG5.6.7.8, MGRN1_CpG12, MGRN1_CpG15, MGRN1_CpG16.17.18, MGRN1_CpG19.20, MGRN1_CpG22.23, MGRN1_CpG26, RAPSN_CpG1, RAPSN_CpG2, RAPSN_CpG4, RAPSN_CpG6, RAPSN_CpG7, RAPSN_CpG8.

In model 2 the antigen CA125, which is commonly used in ovarian cancer diagnostics, was omitted. In model 3, all methylation sites of model 1 and the clinical marker age was included in the model. Model 4 included only methylation markers within the indicated genes. Model 5 used all miRNAs included in model 1 and the clinical marker age, whereas model 6 used only the expression level of the indicated miRNAs. The accuracy, sensitivity, specificity and area under the curve (AUC) of the indicated models is provided in the table below. For prediction random forests and extremely boosted trees were used.

FIG. 3: refers to miRNA markers in ovarian cancer. Two different models and individual miRNA markers tested in the Dresden cohort of ovarian cancer patients. Model 1 used all miRNAs depicted in the above graphs, whereas model 2 used only the indicated miRNAs. The accuracy, sensitivity, specificity and area under the curve (AUC) of the indicated models is provided in the table below.

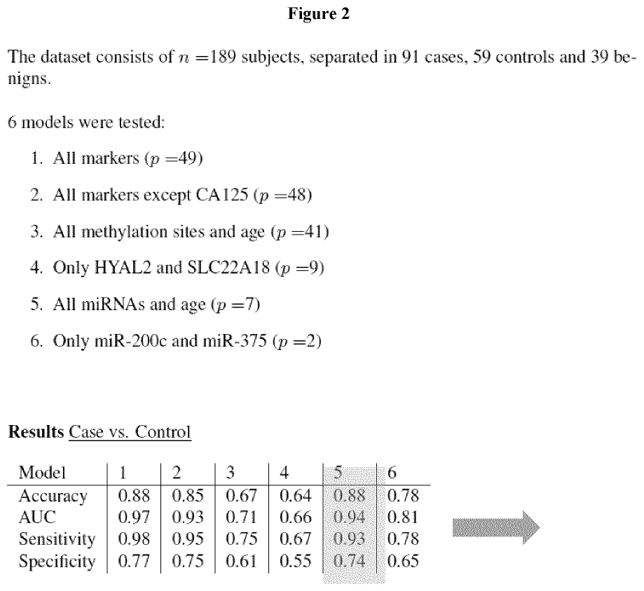

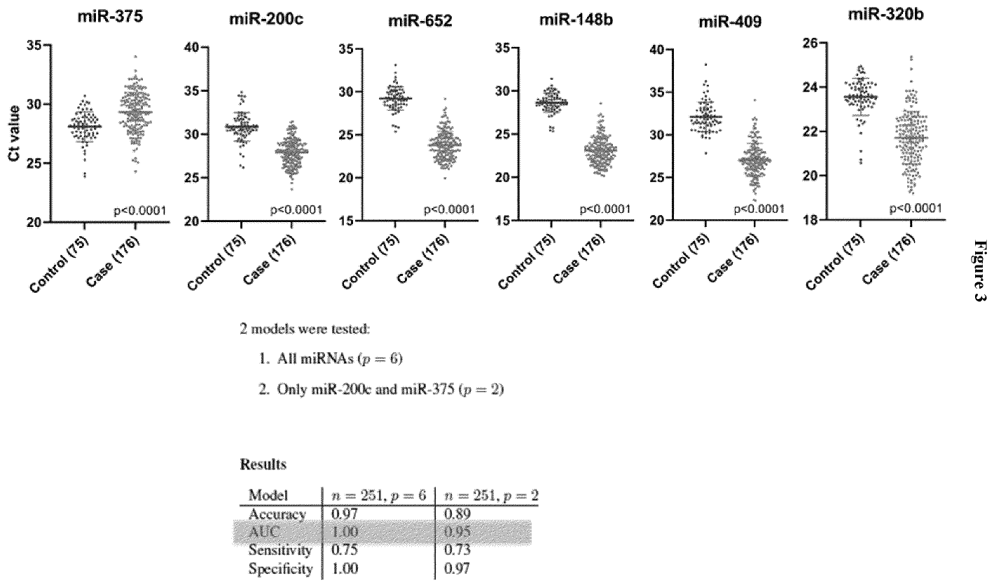

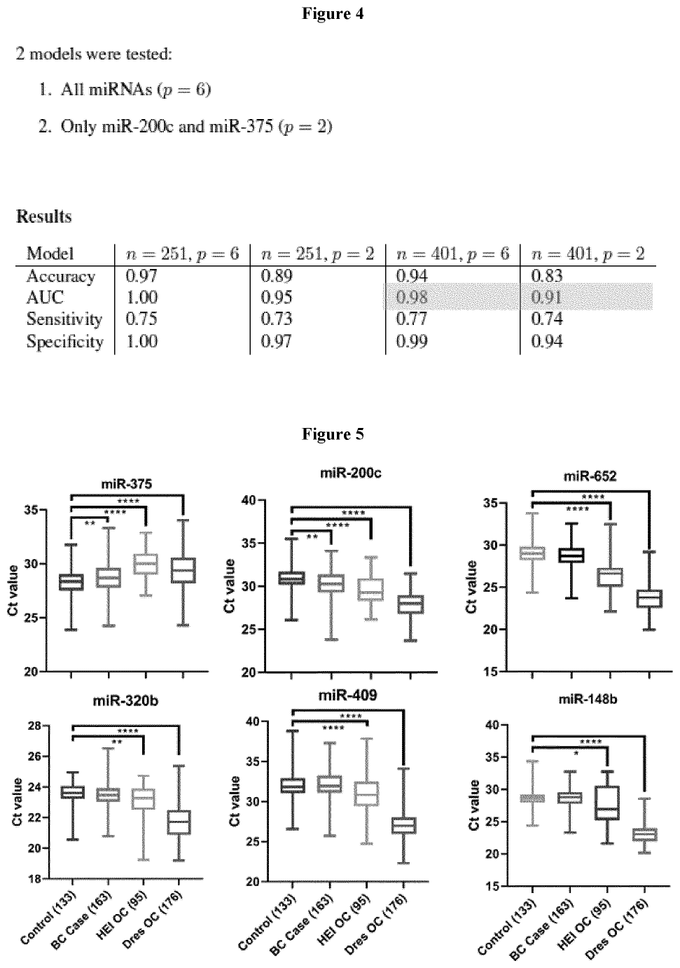

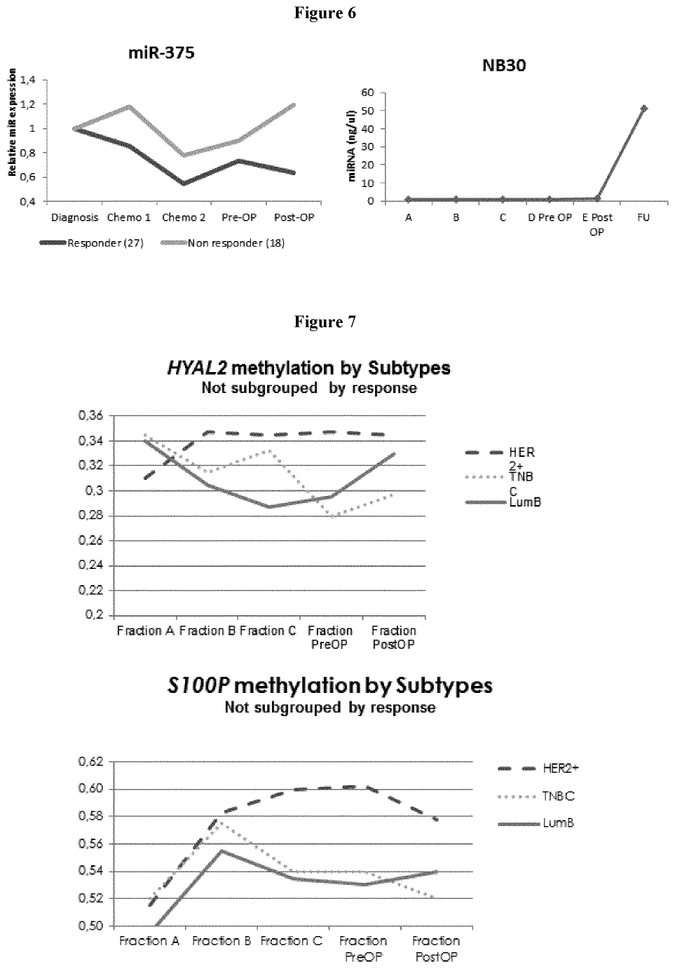

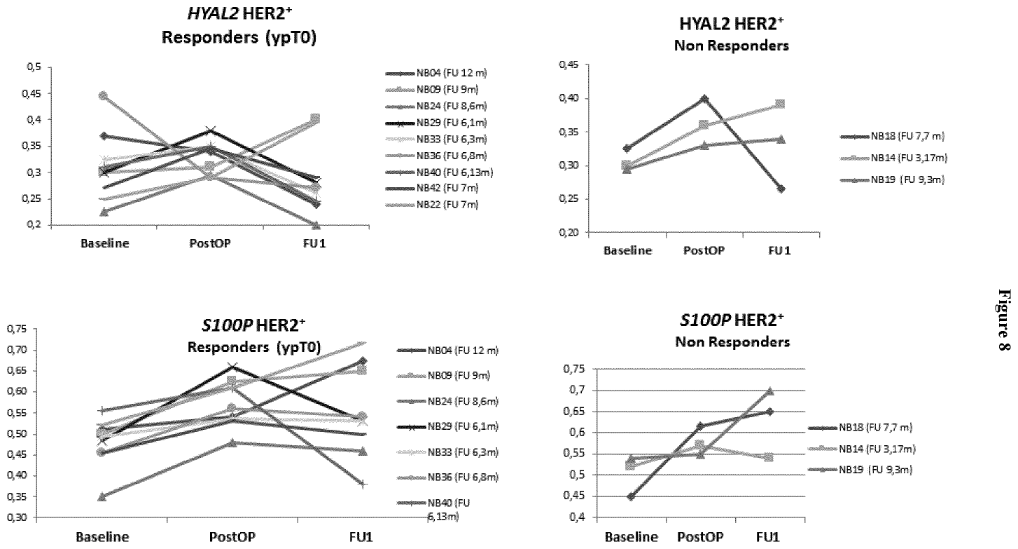

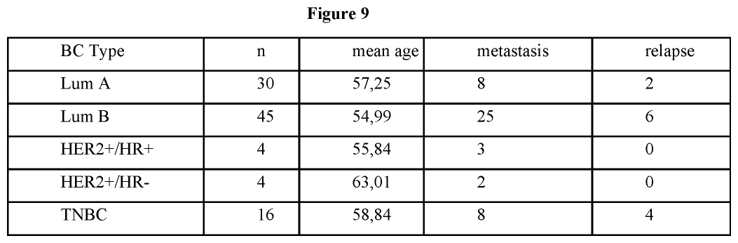

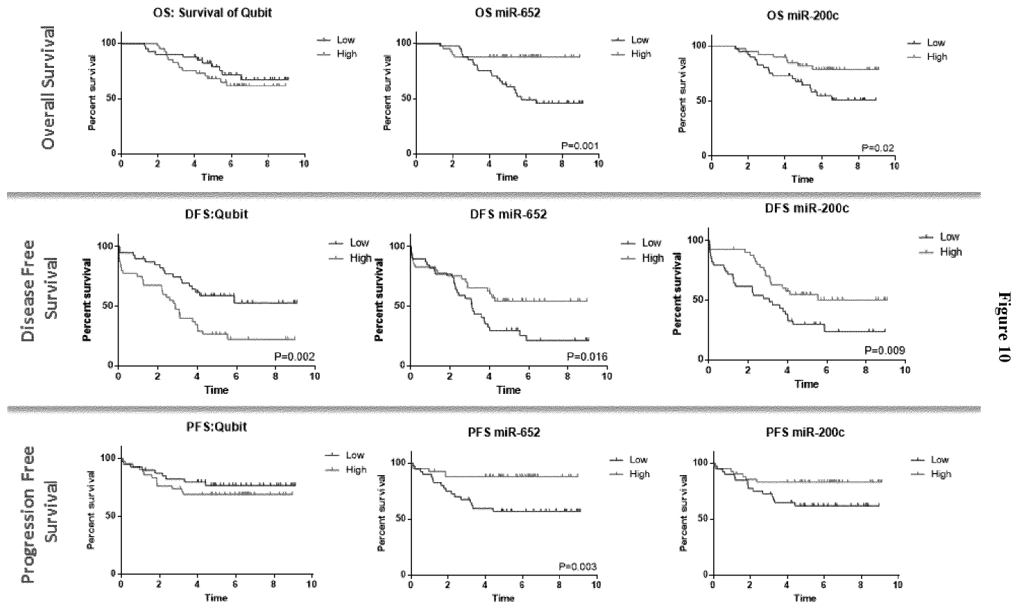

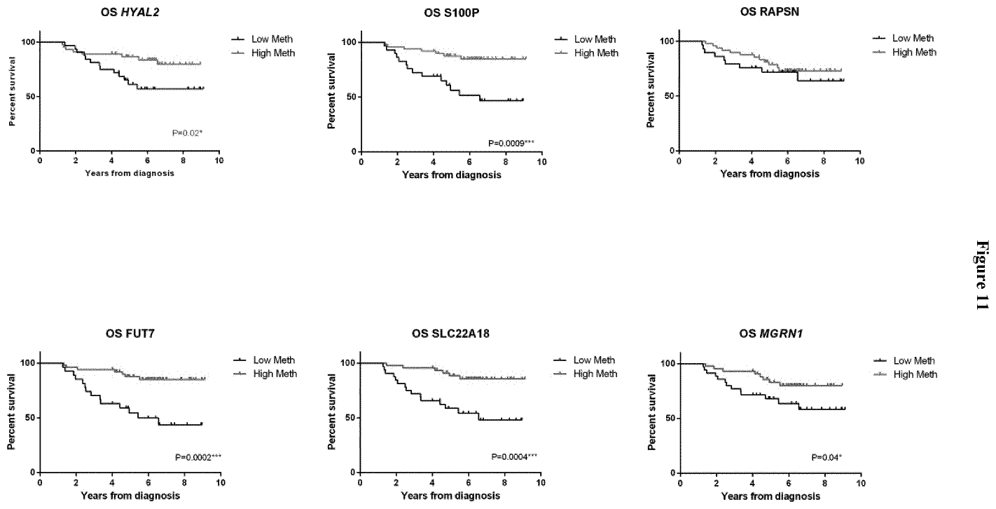

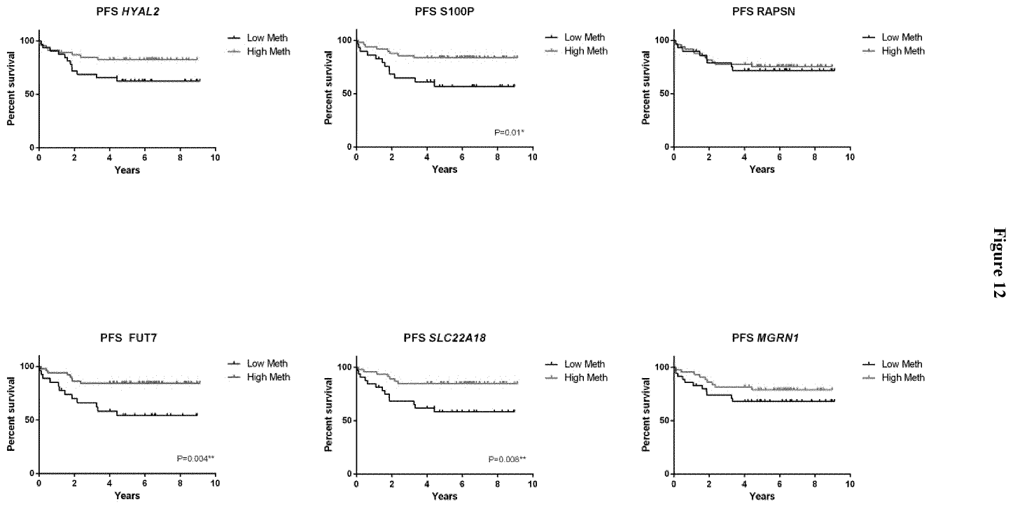

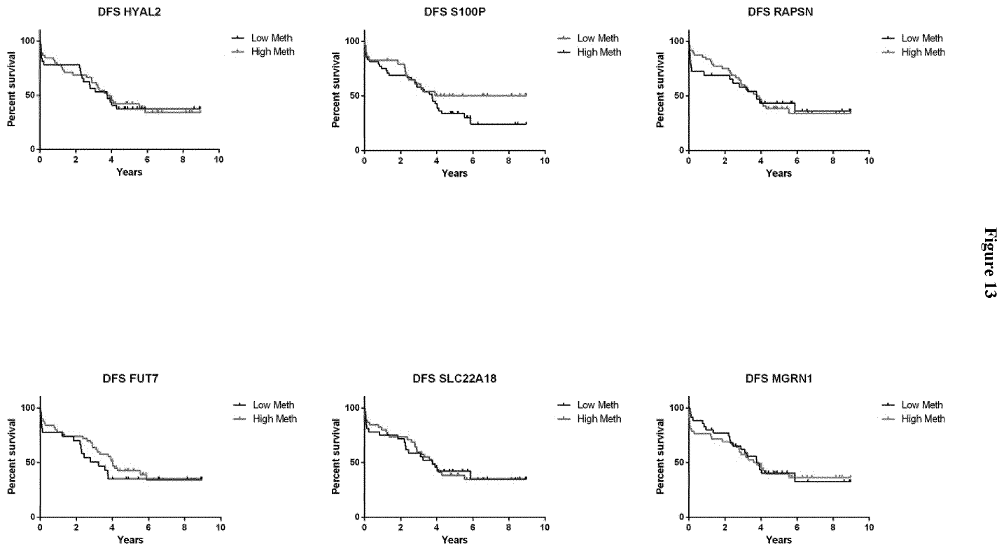

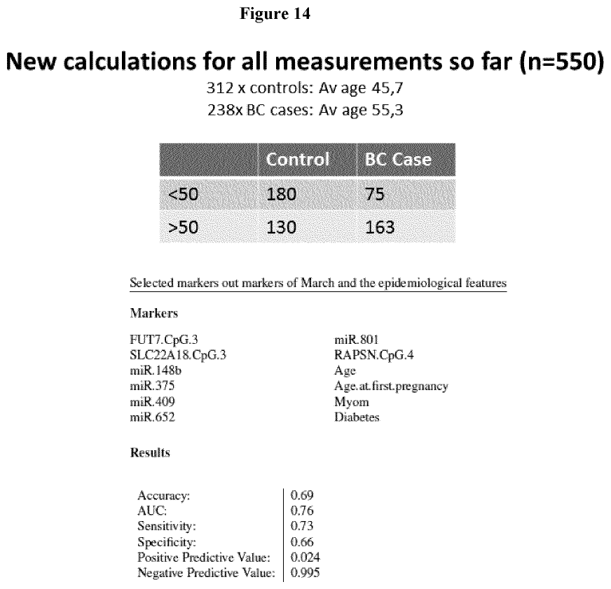

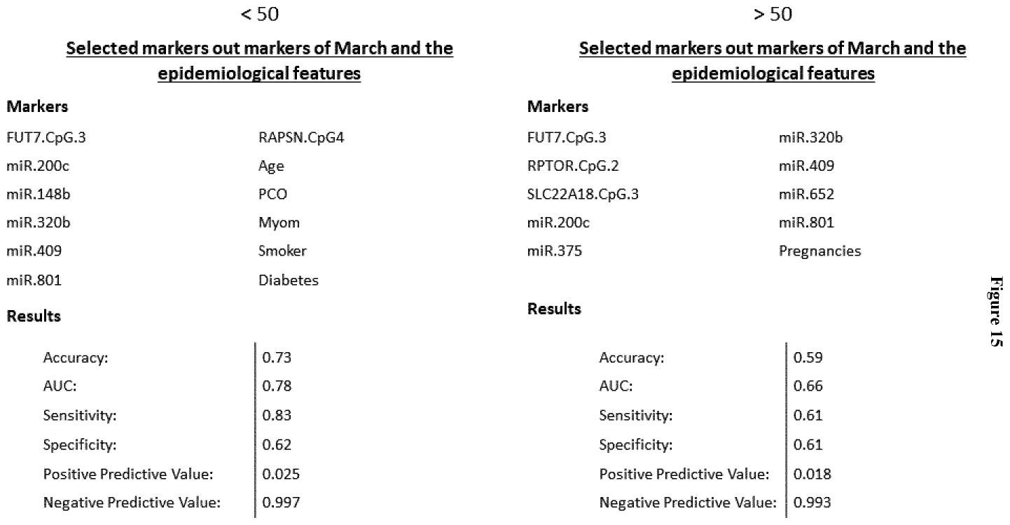

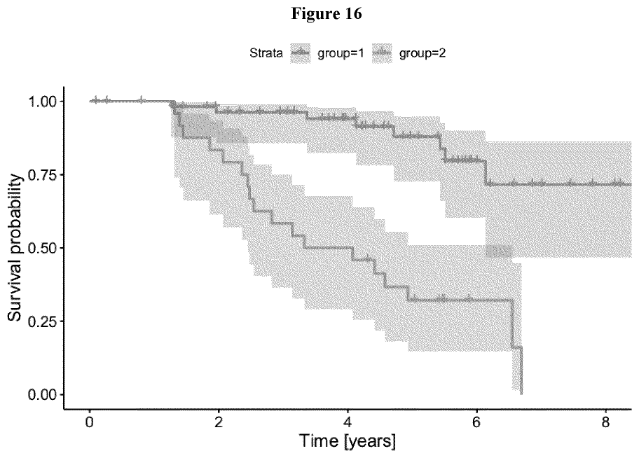

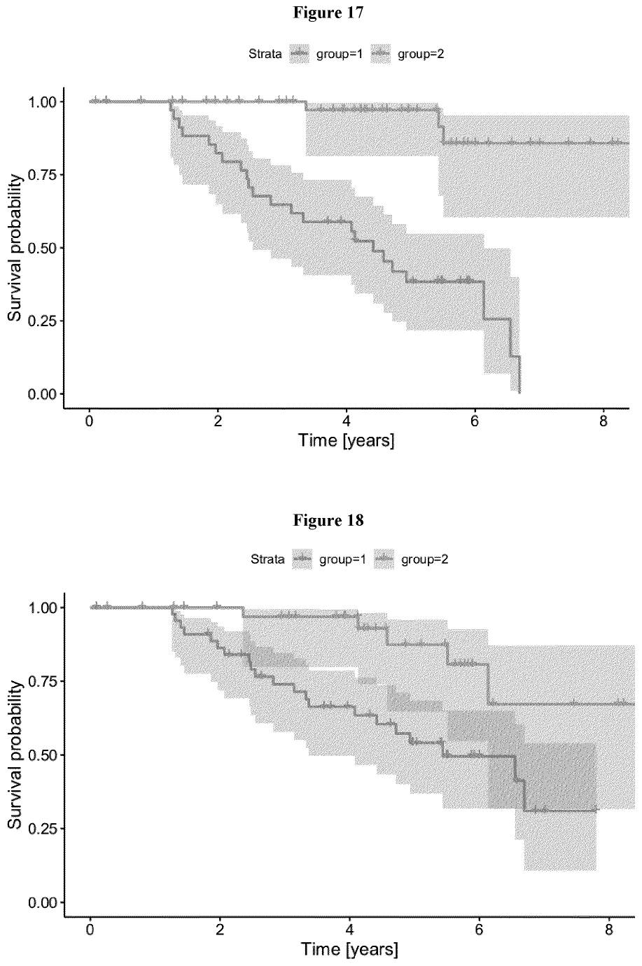

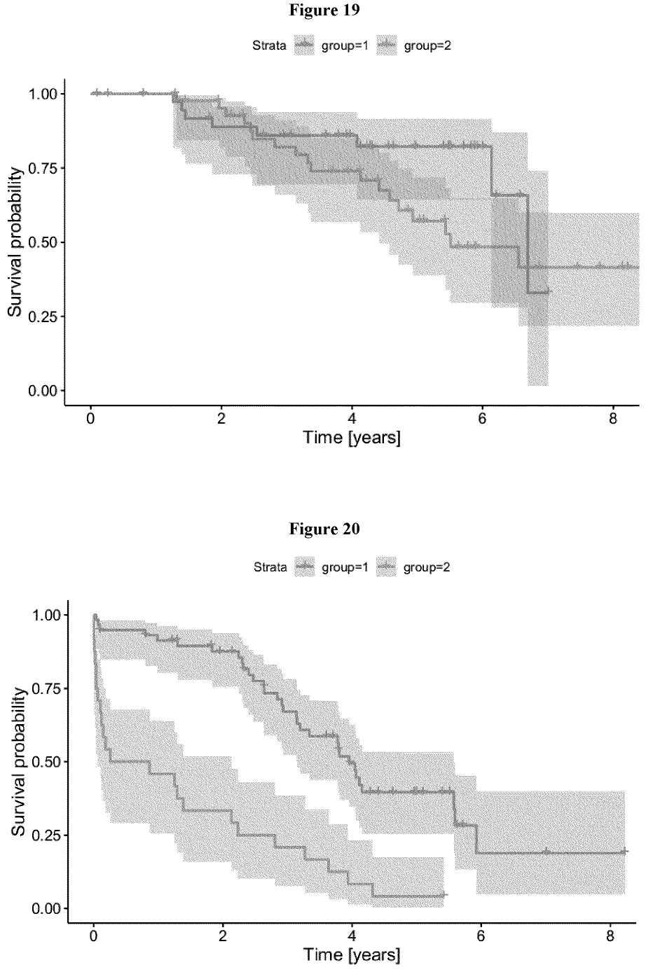

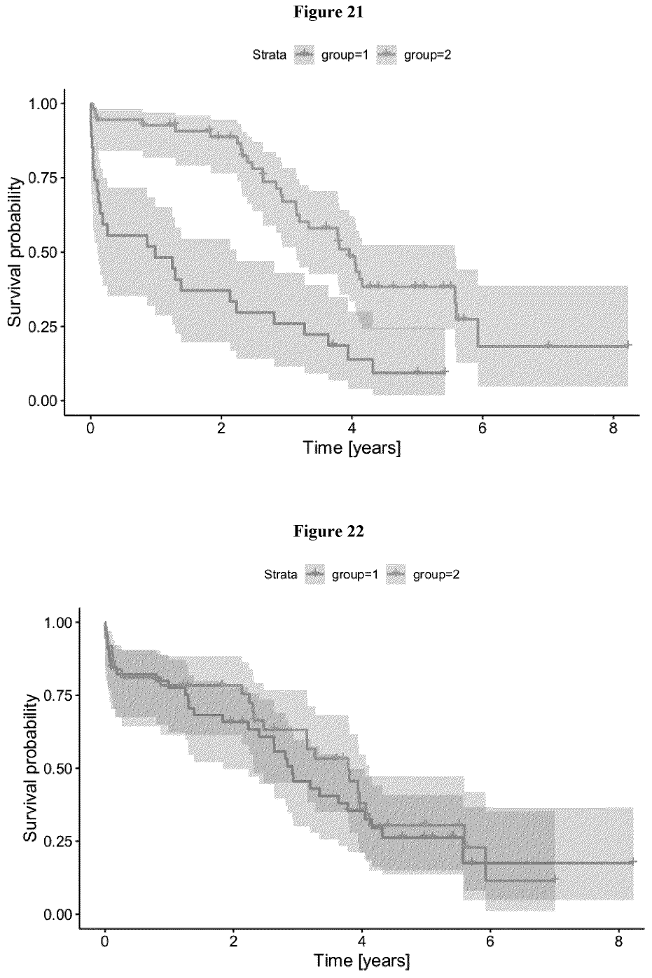

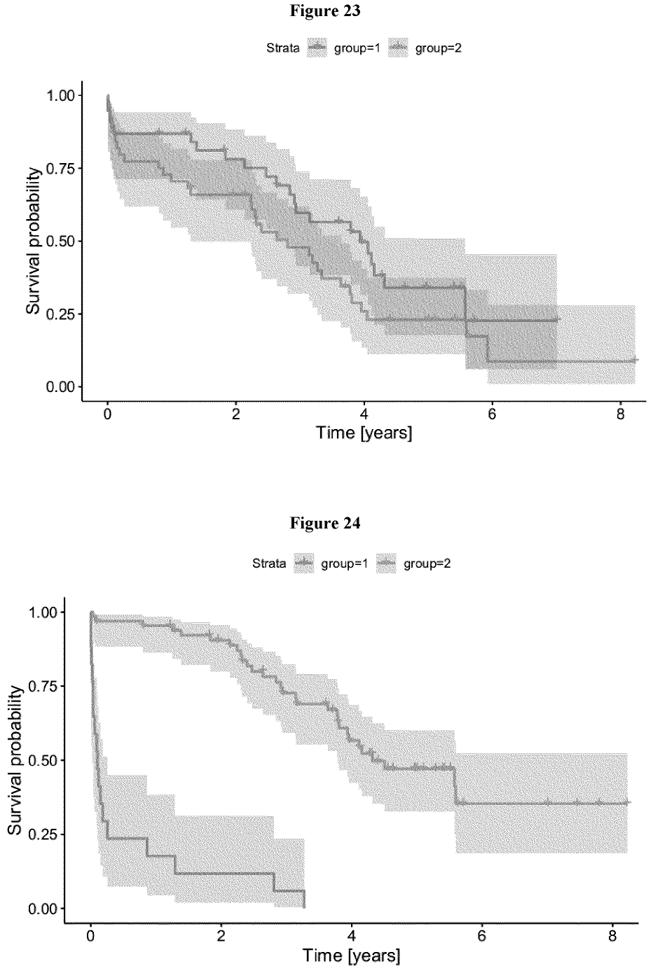

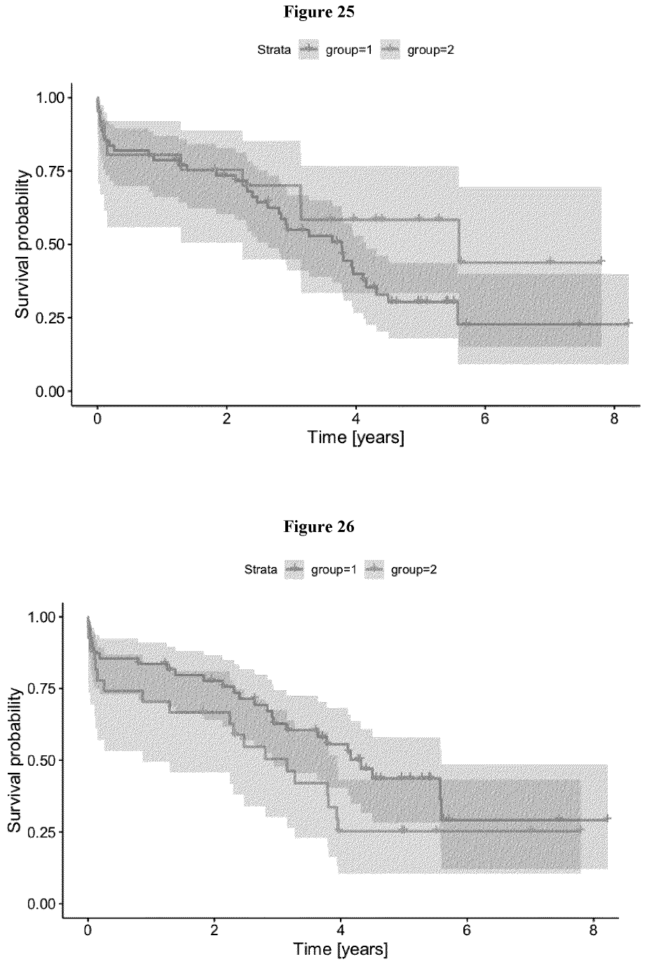

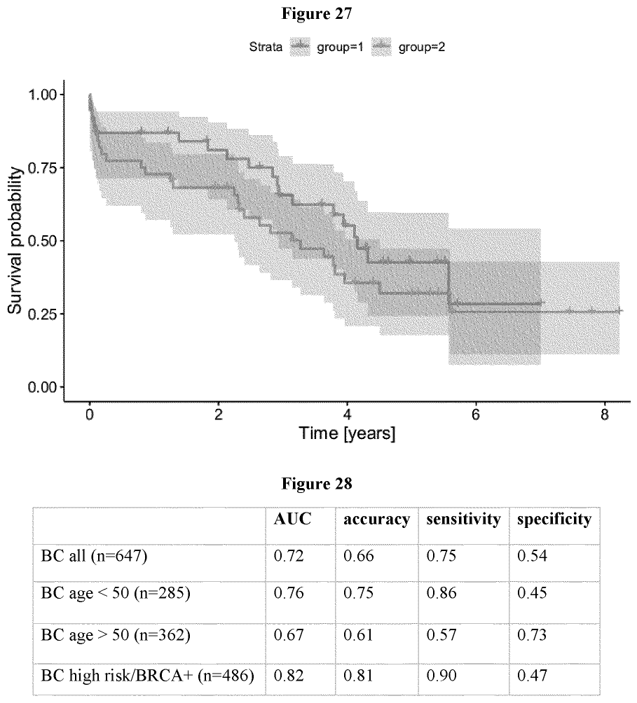

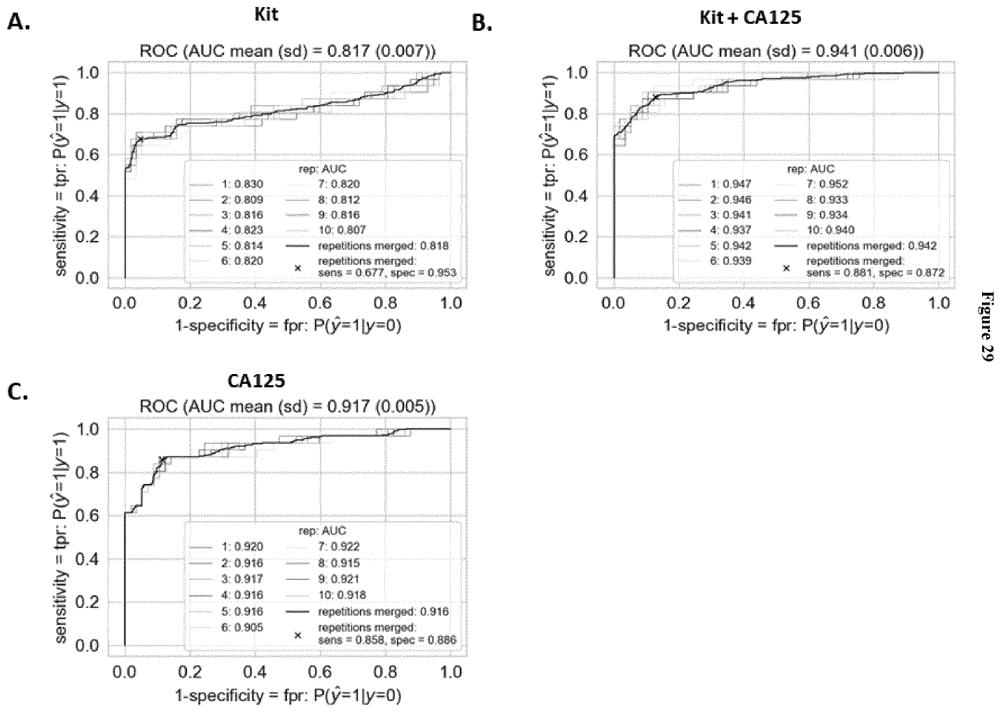

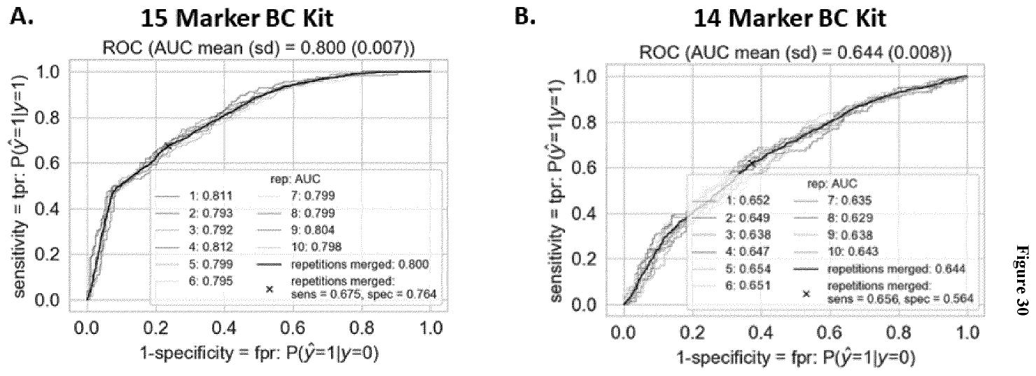

FIG. 4: refers to markers in ovarian cancer. Two models were tested in the combined ovarian cancer cohorts HEIScreen and Dresden of FIGS. 2 and 3. Model 1 used all miRNAs of model 1 of FIG. 3, whereas model 2 used only the indicated miRNAs. The columns indicating 251 tested subjects used the Dresden cohort only (176 cases and 75 controls), whereas the 401 subjects include additional the HEIScreen cohort (91 cases and 59 controls).

FIG. 5: refers to miRNA markers in breast cancer and ovarian cancer. The expression levels of the indicated miRNAs measured in the HEIScreen and Dresden cohort of ovarian cancer patients and 163 breast cancer cases. The asterisks refer to the following p-values, *p<0.05, **p<0.01, ***p<0.001 and ****p<0.0001

FIG. 6: refers to miRNA markers in breast cancer. miRNA-375 was measured in the NeoAdjuvant cohort of breast cancer patients. The expression level of miR-375 correlated with the response of the patients to the cancer treatment and can predict the response to therapy. The left graph depicts miR-375 expression in the NeoAdjuvant cohort. The cohort members have been grouped into responders and non-responders according to their pathological response at time of surgery (OP). The left graph depicts miR-375 expression levels during different stages of treatment. The expression is higher in the subjects not responding to the treatment. The right graph provides data from a representative patient (NB30), which was a non-responder with progressive disease (metastasis) also characterized by a 50-fold increased level of cell-free miRNA (Qubit). A: time of diagnosis; B and C: intervals during neo-adjuvant chemotherapy; D Pre-OP: pre-surgery, E Post OP: 22 days post-surgery, FU: follow-up 7 months after surgery

FIG. 7: refers to methylation markers in breast cancer. The methylation levels of individual CpG sites within the indicated genes were measured. The markers were determined in the Neoadjuvant cohort consisting of 54 patients and were measured at five time points. The y-axis of the graphs refers to methylation percentage. HER2+, TNBC and LumB refer to different types of breast cancer. A: time of diagnosis; B and C: intervals during neo-adjuvant chemotherapy; D Pre-OP: pre-surgery, E Post OP: post-surgery

FIG. 8: refers to methylation markers in HER2+ breast cancer. Individual methylation levels in members of the NeoAdjuvant cohort were determined. The members are grouped into responders and non-responders due to their pathological response determined at time of surgery.

FIG. 9: refers to a long-term follow-up study on breast cancer. The characteristics of the members of the longterm follow-up study is provided. 87 patients where measured at first diagnosis and follow-up samples have been obtained from 52 patients, whereas 5 samples were of bad quality and 26 patients deceased during study.

FIG. 10 refers to miRNAs as prognostic markers in the long-term follow-up study. The miRNAs miR-625 and miR-200c have been determined and correlated with overall survival (OS), disease free survival (DFS) and progression free survival (PFS). As additional marker Qubit was determined. The time indicated on the x-axis is given in years. The patients studied have been assigned to groups low and high based on their difference from the median value of the miRNA expression level, with the low group being below the median value and the high group above the median.

FIG. 11 refers to DNA methylation as prognostic marker in overall survival. The methylation of CpG dinucleotides within the indicated genes has been determined (the same CpGs as listed in figure have been used). The patients have then be assigned to two different groups (low meth and high meth) based on minus 2 standard deviations from the mean methylation of each amplicon. Five of the six tested genes correlate significantly with OS a bad prognosis when having low methylation levels at time of diagnosis.

FIG. 12 refers to DNA methylation as prognostic marker in progression free survival. The methylation of CpG dinucleotides (same as in FIG. 11) within the indicated genes has been determined. The patients have then be assigned to two different groups (low meth and high meth) based on the deviation from the mean for each of the methylation sites. Three of the six tested genes correlate significantly with PFS when having low methylation values at time of diagnosis.

FIG. 13 refers to DNA methylation as prognostic marker in disease-free survival. The methylation of CpG dinucleotides (same as in FIG. 11) within the indicated genes has been determined. The patients have then be assigned to two different groups (low meth and high meth) based on the deviation from the mean for each of the methylation sites.

FIG. 14 refers to a test of markers in the whole cohort of breast cancer cases (312 controls and 238 breast cancer cases) and the best predictor markers as calculated by the elastic net model.

The following markers have been used for the analysis: Qubit, Age, miR-148b, miR-652, miR-200c, miR-409, miR-375, miR-320b, miR-801, HYAL2_CpG1, HYAL2_CpG2, HYAL2_CpG3, HYAL2_CpG4, S100P_CpG2.3, S100P_CpG4, S100P_CpG7, S100P_CpG8, S100P_CpG9, S100P_CpG_10.11.12, SLC22A18_CpG1, SLC22A18_CpG3, SLC22A18_CpG4, SLC22A18_CpG6, SLC22A18_CpG8, RPTOR_CpG1, RPTOR_CpG2, RPTOR_CpG3, RPTOR_CpG5, RPTOR_CpG6, RPTOR_CpG8, RAPSN_CpG1, RAPSN_CpG2, RAPSN_CpG4, RAPSN_CpG5, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, FUT7_CpG8, MGRN1_CpG2, MGRN1_CpG4, MGRN1_CpG_5.6.7.8, MGRN1_CpG12, MGRN1_CpG15, MGRN1_CpG16.17.18, MGRN1_CpG_19.20, MGRN1_CpG_22,23, MGRN1_CpG26, MGRN1_CpG27, MGRN1_CpG28, MGRN1_CpG29, MGRN1_CpG31, MGRN1_CpG32, Nationality, Height [cm], Weight [kg], Age at first period, High Cholesterol, Diabetes, Endometriosis, Myom, Ovarian cyst, PCO, Autoimmune disease, Medication, Pregnancies, Age at first pregnancy, Contraceptives or hormones, Smoker, Vegetarian, Sports

FIG. 15 refers to selected best predictor markers in the same cohort of breast cancer cases as in FIG. 14, but the groups are split in subgroups according to age (<50 years and >50 years).

FIG. 16 refers to a long-term study of the HEIScreen breast cancer cohort referring to overall survival. The following list of markers were used for the long term study (Overall survival, Progression- and Disease-free survival analysis (n=63): age, disease Burden, Breast Cancer Type, Stage, cT, cN, cM, “pT (Surgery)”, “pN (Surgery)”, chemotherapy (yes or no), HYAL2 CpG1, HYAL2_CpG2, HYAL2_CpG3, HYAL2_CpG4, S100P_CpG2.3, S100P_CpG4, S100P_CpG7, S100P_CpG8, S100P_CpG9, S100P_CpG10.11.12, RAPSN_CpG1, RAPSN_CpG2, RAPSN_CpG4, RAPSN_CpG5, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, SLC22A18_CpG1, SLC22A18_CpG3, SLC22A18_CpG4, SLC22A18_CpG6, SLC22A18_CpG8, MGRN1 CpG.2, MGRN1_CpG4, MGRN1 CpG5.6.7.8, MGRN1 CpG12, MGRN1 CpG15, MGRN1 CpG16.17.18, MGRN1 CpG_19.20, MGRN1 CpG_22.23, MGRN1 CpG26, RPTOR_CpG1, RPTOR_CpG2, RPTOR_CpG3, RPTOR_CpG5, RPTOR_CpG6, RPTOR_CpG8, Qubit, miR-148b, -200c, -320b, -375, -409, -652, -205, 141 and -210.

The goal of this study was to compare overall survival (OS), disease-free survival (DFS) and progression-free survival (PFS) and select for each of them markers which allow a good comparison. In the first analysis, the clinical markers Age diagnosis, Disease Burden, Breast Cancer Type, cT, cN, cM, Grade, pT and pN were used beside the markers. In a second analysis, pT and pN were excluded. Furthermore, there are two analyses where only the methylation markers and the miRNAs, respectively, were used.

The markers were selected by fitting an Elastic-Net penalized Cox-model. Then, two groups were built based on the selected markers by 2-means clustering and the corresponding survival times were compared by plotting the Kaplan Meier curves with corresponding confidence intervals.

The markers selected from above marker panel as best predictors for OS were the clinical markers Disease Burden (type of metastasis: visceral, non-visceral or both), Breast Cancer Type, cT, cM and pT. Furthermore, the following methylation markers were selected by the model HYAL2 CpG3, HYAL2 CpG4, S100P CpG9, and RPTOR CpG8 as well as the expression level of mi-200c. A log-rank test yielded a significant difference between group 1 and 2 (p<0.0001).

FIG. 17 refers to the same study as in FIG. 16 but the marker pT and pN (pathological staging of the tumor) was excluded. A log-rank test yielded a significant difference between group 1 and 2 (p<0.0001).

FIG. 18 refers to the same study as in FIG. 16 but only the methylation markers were used (n=43). The model then selected the best markers which are: HYAL2 CpG3, HYAL2 CpG4, S100P CpG9, RAPSNCpG4, RAPSN CpG5, FUT7 CpG3, SLC22A18 CpG8, MGRN1CpG2, MGRN1CpG4 and RPTOR CpG8. A log-rank test yielded p=0.003.

FIG. 19 refers to the same study as in FIG. 16, but all miRNAs (miR-148b, -200c, -320b, -375, -409, -652, -205, 141 and -210) were included in the analysis. Due to the low number of variables no miRNA could be selected, A log-rank test yielded p=0.136.

FIG. 20 refers to the same study as detailed in FIG. 16 with the difference that in this instance disease-free survival was correlated with the markers indicated in FIG. 16. The markers selected for this test as best predictors were the clinical markers ‘disease burden’, ‘breast cancer type’, cT, cN, and cM. Furthermore, the following methylation markers were selected by the model: SLC22A18 CpG8 and MGRN1 CpG2 as well as Qubit and the miRNAs 200c, 320b, and -141. A log-rank test yielded a significant difference between group 1 and 2 (p<0.001).

FIG. 21 refers to the same study as FIG. 20 using disease-free survival as parameter to be predicted by the model used. The same markers as indicated in FIG. 20 were used with the exception of pT and pN. The model selected the following markers as best predictors: Disease burden, breast cancer type, cT, cN, cM, SLC22A18.CpG8, MGRN1.1.CpG2, miR-200c, -320b, -141 and Qubit. A log-rank test yielded a significant difference between group 1 and 2 (p<0.0001).

FIG. 22 refers to the same study as FIG. 20. However, the markers used for this model only include the methylation markers. The following methylation markers were selected as the best predictors: HYAL2 (CpG2 and CpG 4), S100P (CpG2.3 and CpG4), RAPSN (CpG7 and CpG8), FUT7 (CpG3), SLC22A18 (CpG4 and CpG8), MGRN1 (CpG2 and CpG26) and RPTOR (CpG5 and CpG8). A log-rank test yielded p=0.524.

FIG. 23 refers to the same study as FIG. 20, but only miRNA markers were used The used elastic net model could not predict best markers due to the low number of variables (miRNA markers). A log-rank test yielded p=0.108.

FIG. 24 refers to the same study as FIG. 16 and used all markers indicated there, with the difference that in this instance progression-free survival was correlated with the markers. The markers selected as best predictors were the clinical markers ‘disease burden’, cN and cM. Furthermore, the following methylation markers were selected by the model HYAL2 CpG4, S100P CpG4, RAPSN CpG7, FUT7 CpG3, SLC22A18 CpG8, and MGRN1 CpG2 as well as Qubit and the miRNAs-200c, -320b, and -375. A log-rank test yielded a significant difference between group 1 and 2 (p<0.001).

FIG. 25 refers to the same study as FIG. 24 using progression-free survival as parameter to be predicted by the model used. The same markers as indicated in FIG. 24 were used with the exception of pT and pN and the model selected the same best markers as in FIG. 24A log-rank test yielded p=0.174.

FIG. 26 refers to the same study as FIG. 24, but only methylation markers were tested. The following methylation markers were selected by the model: HYAL2 CpG3 and CpG4, RAPSN CpG2, CpG5, and CpG7, FUT7 CpG3, SLC22A18 CpG8, MGRN1 CpG2 and CpG26 and RPTOR CpG5 and CpG8. A log-rank test yielded p=0.108.

FIG. 27 refers to the same study as FIG. 24, but only miRNA markers were tested. Only the miRNA-markers miR-148b, -200c, -320b, -375, -409, -652, -205, 141 and -210 were included in the analysis. Due to the low number of variables no miRNA could be selected. A log-rank test yielded p=0.23.

FIG. 28 refers to diagnosis of breast cancer (BC) in general (all) and in patient subgroups, grouped by age or high risk groups (BRCA+). The following methylation markers were determined for the high risk group: HYAL2_CpG1, HYAL2_CpG2, HYAL2_CpG3, HYAL2_CpG4, S100P_CpG2,3, S100P_CpG7, S100P_CpG8, S100P_CpG9, S100P_CpG10,11,12, SLC22A18_CpG1, SLC22A18_CpG3, SLC22A18_CpG4, SLC22A18_CpG6, RPTOR_CpG1, RPTOR_CpG2, RPTOR_CpG3, RPTOR_CpG5, RPTOR_CpG6, RPTOR_CpG8, RAPSN_CpG1, RAPSN_CpG4, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, MGRN1_CpG4, MGRN1_CpG5,6,7,8, MGRN1_CpG12, MGRN1_CpG15, MGRN1_CpG16,17,18, MGRN1_CpG19,20, MGRN1_CpG22,23, MGRN1_CpG26.

The following markers have been determined for the BC all group and the age subgroups: Age, Height [cm], Weight [kg], Diabetes, Myom, Autoimmune disease, Pregnancies, Age at first pregnancy, Contraceptives, Smoker, Sports, miR-148b, miR-652, miR-200c, miR-409, miR-375, miR-320b, Qubit, HYAL2_CpG_1, HYAL2_CpG_2, HYAL2_CpG_3, HYAL2_CpG_4, S100P_CpG_2,3, S100P_CpG_4, S100P_CpG_7, S100P_CpG_8, S100P_CpG_9, S100P_CpG_10,11,12, SLC22A18_CpG_1, SLC22A18_CpG_3, SLC22A18_CpG_4, SLC22A18_CpG_6, SLC22A18_CpG_8, RPTOR_CpG_1, RPTOR_CpG_2, RPTOR_CpG_3, RPTOR_CpG_5, RPTOR_CpG_6, RPTOR_CpG_8, RAPSN_CpG_1, RAPSN_CpG_2, RAPSN_CpG_4, RAPSN_CpG_5, RAPSN_CpG_6, RAPSN_CpG_7, RAPSN_CpG_8, FUT7_CpG_1, FUT7_CpG_2, FUT7_CpG_3, FUT7_CpG_4, FUT7_CpG_6, FUT7_CpG_7, MGRN1_CpG_2, MGRN1_CpG_4, MGRN1_CpG_5,6,7,8, MGRN1_CpG_12, MGRN1_CpG_15, MGRN1_CpG_16,17,18, MGRN1_CpG_19,20, MGRN1_CpG_22,23, MGRN1_CpG_26.

FIG. 29 refers to diagnostic performance in early stage OC. 10-fold cross validation analysis depicts the mean AUC obtained for the OC kit markers alone (A), OC kit markers in combination with CA-125 (B) or CA-125 alone (C) in early stage OC.

FIG. 30 refers to diagnostic performance in breast cancer. 10-fold cross validation analysis depicts the mean AUC obtained for the 15 Marker BC Kit (A) or the 14 Marker BC Kit without age as variable (B) in BC.

List of sequences
hsa-miR-652-3p (MIMAT0003322):
SEQ ID NO: 1
aauggcgccacuaggguugug
hsa-miR-652-5p (MIMAT0022709):
SEQ ID NO: 2
caacccuaggagagggugccauuca
hsa-miR-409-3p (MIMAT0001639):
SEQ ID NO: 3
gaauguugcucggugaaccccu
hsa-miR-409-5p (MIMAT0001638):
SEQ ID NO: 4
agguuacccgagcaacuuugcau
hsa-miR-148b-3p (MIMAT0000759):
SEQ ID NO: 5
ucagugcaucacagaacuuugu
hsa-miR-148b-5p (MIMAT0004699):
SEQ ID NO: 6
aaguucuguuauacacucaggc
hsa-miR-200c-3p (MIMAT0000617):
SEQ ID NO: 7
uaauacugccggguaaugaugga
hsa-miR-200c-5p (MIMAT0004657):
SEQ ID NO: 8
cgucuuacccagcaguguuugg
hsa-miR-375-3p (MIMAT0000728):
SEQ ID NO: 9
uuuguucguucggcucgcguga
hsa-miR-375-5p (MIMAT0037313):
SEQ ID NO: 10
gcgacgagccccucgcacaaacc
hsa-miR-141-3p (MIMAT0000432):
SEQ ID NO: 11
uaacacugucugguaaagaugg
hsa-miR-141-5p (MIMAT0004598):
SEQ ID NO: 12
caucuuccaguacaguguugga
hsa-miR-451a (MIMAT0001631):
SEQ ID NO: 13
aaaccguuaccauuacugaguu
hsa-mir-320b-1 (MI0003776):
SEQ ID NO: 14
auaaauuaaucccucucuuucuaguucuuccuagagugaggaaaagcuggg
uugagagggcaaacaaauuaa
hsa-mir-320b-2 (MI0003839):
SEQ ID NO: 15
gucucuuaggcuuucucuucccagauuucccaaaguugggaaaagcugggu
ugagagggcaaaaggaaaaa

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

Several documents are cited throughout the text of this specification. Each of the documents cited herein (including all patents, patent applications, scientific publications, manufacturers' specifications, instructions etc.), whether supra or infra, is hereby incorporated by reference in its entirety. Nothing herein is to be construed as an admission that the invention is not entitled to antedate such disclosure by virtue of prior invention.

MiRNAs are small, non-coding RNAs (˜18-25 nucleotides in length) that regulate gene expression on a post-transcriptional level by degrading mRNA molecules or blocking their translation (Bartel D P. Cell 2004; 116: 281-97). Hence, they play an essential role in the regulation of a large number of biological processes, including cancer (Calin et al., Proc Natl Acad Sci USA 2002; 99:15524-9). Under the standard nomenclature system, names are assigned to experimentally confirmed miRNAs. The prefix “mir” is followed by a dash and a number. The uncapitalized “mir-” refers to the pre-miRNA, while a capitalized “miR-” refers to the mature form. MiRNAs with nearly identical sequences bar one or two nucleotides are annotated with an additional lower case letter. Species of origin is designated with a three-letter prefix, e.g. hsa for Homo sapiens (human). Two mature miRNAs originating from opposite arms of the same pre-miRNA are denoted with a -3p or -5p suffix.

Circulating miRNAs are defined as miRNAs present in the cell-free component of body fluids like plasma, serum, and the like. Lawrie et al. (Br J Haematol 2008; 141:672-5) were among the first to demonstrate the presence of miRNAs in bodily fluids. Since then, circulating miRNAs have been reported as aberrantly expressed in blood plasma or serum in different types of cancer, e.g. prostate, colorectal or esophageal carcinoma (Brase et al., Int J Cancer 2011; 128:608-16; Huang et al., Int J Cancer 2010; 127:118-26; Zhang et al., Clin Chem 2010; 56:1871-9). Their most important advantages include the possibility to be measured repeatedly in a minimally invasive manner as well as their remarkable stability in plasma/serum, where they circulate mostly outside of exosomes and are stable due to their binding to Argonaute proteins (Mitchell et al., Proc Natl Acad Sci USA 2008; 105:10513-8; Turchinovich et al. Nucleic Acids Res 2011; 39:7223-33; Arroyo et al., Proc Natl Acad Sci USA 2011; 108:5003-8).

As used herein, the term “microRNA” and variations such as “miRNA” and “miR” is understood by the skilled artisan and relates to a short ribonucleic acid (RNA) molecule found in eukaryotic cells and in body fluids of metazoan organisms. MiRNA include human miRNAs, mature single stranded miRNAs, precursor miRNAs (pre-miR), and variants thereof, which may be naturally occurring. In some instances, the term “miRNA” also includes primary miRNA transcripts (pri-miRNAs) and duplex miRNAs. Unless otherwise noted, when used herein, the name of a specific miRNA refers to the mature miRNA. MiRNA-precursor may consists of 25 to several thousand nucleotides, typically 40 to 130, 50 to 120, or 60 to 110 nucleotides. Typically, a mature miRNA consists of 5 to 100 nucleotides, often 10 to 50, 12 to 40, or 18 to 26 nucleotides. The term miRNA also includes the “guide” strand which eventually enters the RNA-induced silencing complex (RISC) as well as to the “passenger” strand complementary thereto.

The sequence of several miRNAs is known in the art and readily assessable to the skilled person via well-known sequence databases, such as e.g. miRBase (http://www.mirbase.org/), (Griffiths-Jones S., NAR 2004 32 (Database Issue): D109-D111; Kozomara A, Griffiths-Jones S., NAR 2011 39 (Database Issue): D152-D157). It is understood that below indicated database accession numbers of the individual miRNAs are those of miRNAs of human origin. However these database entries also provide the database accession numbers of the respective miRNA of different origin, such as e.g. miRNAs of any mammal, reptile, or bird origin, such as e.g. those selected from the group consisting of laboratory animals (e.g. mouse or rat), domestic animals (including e.g. guinea pig, rabbit, horse, donkey, cow, sheep, goat, pig, chicken, camel, cat, dog, turtle, tortoise, snake, or lizard), or primates including chimpanzees, bonobos, and gorillas miRNA. It is also understood that the reference to a specific miRNA by its number (e.g. miR-652) equally refers to the -3p and -5p sequence (miR-652-3p and miR-652-5p).

The sequence of miR-652 is deposited at miRBase ID MI0003667 which comprises hsa-miR-652-3p (MIMAT0003322) and hsa-miR-652-5p (MIMAT0022709), which corresponds to SEQ ID NO: 1 and 2, respectively, of the present invention.

The sequence of miR-409 is deposited at miRBase ID MI0001735, which comprises hsa-miR-409-3p (MIMAT0001639) and hsa-miR-409-5p (MIMAT0001638), which corresponds to SEQ ID NO: 3 and 4, respectively, of the present invention.

The sequence of miR-148b is deposited at miRBase ID M10000811, which comprises hsa-miR-148b-3p (MIMAT0000759) and hsa-miR-148b-5p (MIMAT0004699), which corresponds to SEQ ID NO: 5 and 6, respectively, of the present invention.

The sequence of miR-200c is deposited at miRBase ID MI0000650, which comprises hsa-miR-200c-3p (MIMAT0000617) and hsa-miR-200c-5p (MIMAT0004657), which corresponds to SEQ ID NO 7 and 8, respectively, of the present invention.

The sequence of miR-375 is deposited at miRBase ID MI0000783, which comprises hsa-miR-375-3p (MIMAT0000728) and hsa-miR-375-5p (MIMAT0037313), which corresponds to SEQ ID NO 9 and 10, respectively, of the present invention.

The sequence of miR-141 is deposited at miRBase ID MI0000457, which comprises hsa-miR-141-3p (MIMAT0000432) and hsa-miR-141-5p (MIMAT0004598), which corresponds to SEQ ID NO 11 and 12, respectively, of the present invention.

The sequence of miR-451a is deposited at miRBase ID MI0001729, which comprises hsa-miR-451a (MIMAT0001631), which corresponds to SEQ ID NO 13 of the present invention.

The sequence of miR-320b is deposited at miRBase ID MIMAT0005792, which comprises hsa-mir-320b-1 (MI0003776) and hsa-mir-320b-2 (MI0003839), which correspond to SEQ ID 14 and 15, respectively of the present invention.

The term “combination of miRNAs” relates to combinations of the miRNAs of the present invention. The amount of a miRNA can be determined in a sample of a subject by techniques well known in the art. Depending on the nature of the sample, the amount may be determined by PCR based techniques for quantifying the amount of a polynucleotide or by other methods like mass spectrometry or (next generation) sequencing or one of the methods described in the examples (Cissell K A, Deo S K. Trends in microRNA detection. Anal Bioanal Chem. 2009; 394(4):1109-1116 or de Planell-Saguer M, Rodicio M C. Analytical aspects of microRNA in diagnostics: a review. Anal Chim Acta 2011 Aug. 12; 699(2):134-52). The term “determining the amounts of at least the miRNAs of a combination of miRNAs”, as used herein, preferably relates to determining the amount of each of the miRNAs of the combination separately in order to be able to compare the amount of each miRNA of the combination to a reference specific for said miRNA.

The term “primer” as used herein refers to a single-strand oligonucleotide which typically serves as a starting point for DNA-replicating enzymes. A primer binds to or hybridises with a DNA template and typically comprises a sequence being complementary to the DNA sequence to which it is supposed to bind. A primer may also comprise additional sequences e.g. sequences serving as nuclease cleavage sites (e.g. Bam H1, Hind III, etc.). The length of a primer is chosen depending on the intended use. For instance, primers used for the amplification of DNA in Polymerase-Chain Reactions (PCR) typically have a length of at least 10 nucleotides, preferably between 10 to 50 (i.e. 15, 16, 17, 18, 19, 20, 21, 22, 23, 24, 25, 26, 27, 28, 29, 30, 31, 32, 33, 34, 35, 36, 37, 38, 39, 40, 45, 50) nucleotides, more preferably between 15 and 30 nucleotides. Shorter primers of at least 5 nucleotides are used for sequencing of DNA templates. Also, encompassed in the term “primer” are “degenerate primers” which are a mixture of similar but not identical primers. A primer may be tagged or labelled with a marker molecule detectable by spectroscopic, photochemical, biochemical, immunochemical, chemical, or other physical means.

The term “expression level” refers to the amount of gene product present in the body or a sample at a certain point of time. The expression level can e.g. be measured/quantified/detected by means of the protein or mRNA expressed from the gene. The expression level can for example be quantified by normalizing the amount of gene product of interest present in a sample with the total amount of gene product of the same category (total protein or mRNA) in the same sample or a reference sample (e.g. a sample taken at the same time from the same individual or a part of identical size (weight, volume) of the same sample) or by identifying the amount of gene product of interest per defined sample size (weight, volume, etc.). The expression level can be measured or detected by means of any method as known in the art, e.g. methods for the direct detection and quantification of the gene product of interest (such as mass spectrometry) or methods for the indirect detection and measurement of the gene product of interest that usually work via binding of the gene product of interest with one or more different molecules or detection means (e.g. primer(s), probes, antibodies, protein scaffolds) specific for the gene product of interest. The determination of the level of gene copies comprising also the determination of the absence or presence of one or more fragments (e.g. via nucleic acid probes or primers, e.g. quantitative PCR, Multiplex ligation-dependent probe amplification (MLPA) PCR) is also within the knowledge of the skilled artisan.

The GeneBank release the below accession numbers refer to is release 228.

### HYAL2:

Genbank Acc No: NM_003773.5 (GI:289802998) for transcript variant 1,
Genbank Acc No: NM_033158.4 (GI:289802999) for transcript variant 2,
Genbank Acc No: NP_003764.3 (GI:15022801), for the HYAL2 polypeptide encoded by transcript variant 1,
Genbank Acc No: NP_149348.2 (GI:34304377), for the HYAL2 polypeptide encoded by transcript variant 2.

### MGRN1:

Genbank Acc No: NM_001142289.2 for the transcript variant 2, and
Genbank Acc No: NM_001142290.2 for the transcript variant 3, and
Genbank Acc No: NM_001142291.2 for the transcript variant 4, and
Genbank Acc No: NM_015246.3 for the transcript variant 1, and
Genbank Acc No: NP_001135761.2 for the MGRN1 polypeptide encoded by the transcript variant 2;
Genbank Acc No: NP_001135762.1 for the MGRN1 polypeptide encoded by the transcript variant 3;
Genbank Acc No: NP_001135763.2 for the MGRN1 polypeptide encoded by the transcript variant 4;
Genbank Acc No: NP_056061.1 for the MGRN1 polypeptide encoded by the transcript variant 1;

### RPTOR

Genbank Acc No: NM_001163034.1 for the transcript variant 2,
Genbank Acc No: NM_020761.3 for the transcript variant 1
Genbank Acc No: NP_001156506.1 for the RPTOR polypeptide encoded by the transcript variant 2;
Genbank Acc No: NP_065812.1 for the RPTOR polypeptide encoded by the transcript variant 1;

### SLC22A18

Genbank Acc No: NM_002555.5 for the transcript variant 1,
Genbank Acc No: NM_183233.2 for the transcript variant 2,
Genbank Acc No: NP_002546.3 for the SLC22A18 polypeptide encoded by the transcript variant 1;
Genbank Acc No: NP_899056.2 for the SLC22A18 polypeptide encoded by the transcript variant 2;

### FUT7

Genbank Acc No: NM_004479.3 for the transcript,
Genbank Acc No: NP_004470.1 for the FUT7 polypeptide encoded by the transcript;

### RAPSN

Genbank Acc No: NM_005055.5 for the transcript variant 1,
Genbank Acc No: NM_032645.4 for the transcript variant 2,
Genbank Acc No: NP_005046.2 for the RAPSN polypeptide encoded by the transcript variant 1;
Genbank Acc No: NP_116034.2 for the RAPSN polypeptide encoded by the transcript variant 1;

### S100P

Genbank Acc No: NM_005980.3 for the transcript,
Genbank Acc No: NP_005971.1 for the S100P polypeptide encoded by the transcript;

The term “tissue” as used herein, refers to an ensemble of cells of the same origin which fulfil a specific function concertedly. Examples of a tissue include but are not limited to connective tissue, muscle tissue, nervous tissue, and epithelial tissue. Multiple tissues together form an “organ” to carry out a specific function. Examples of an organ include but are not limited to glands, muscle, blood, brain, heart, liver, kidney, stomach, skeleton, joint, and skin.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to traumatic diseases, inflammatory diseases, infectious diseases, cutaneous conditions, endocrine diseases, intestinal diseases, neurological disorders, joint diseases, genetic disorders, autoimmune diseases, and various types of cancer.

“Cancer” refers to a proliferative disorder involving abnormal cell growth which may invade or spread to other tissues or organs of a subject. Cancers are classified by the type of cell that the tumor cells resemble and are therefore presumed to be the origin of the tumor. These types include but are not limited to carcinoma (cancers derived from epithelial cells) sarcoma (cancers arising from connective tissue such as e.g. bone, cartilage, fat, nerve), lymphoma and leukemia (cancer arising from hematopoietic cells that leave the marrow and tend to mature in the lymph nodes and blood), germ cell tumor (cancers derived from pluripotent cells), and blastoma (cancers derived from immature “precursor” cells or embryonic tissue). In particular, cancer includes but is not limited to acute lymphoblastic leukemia (ALL), acute myeloid leukemia, adrenocortical carcinoma, AIDS-related cancers, AIDS-related lymphoma, anal cancer, appendix cancer, astrocytoma, childhood cerebellar or cerebral cancer, basal-cell carcinoma, bile duct cancer, extrahepatic, bladder cancer, bone tumor, osteosarcoma/malignant fibrous histiocytoma, brainstem glioma, brain cancer, brain tumor (cerebellar astrocytoma, cerebral astrocytoma/malignant glioma, ependymoma, medulloblastoma, supratentorial primitive neuroectodermal tumors, visual pathway and hypothalamic glioma), breast cancer, bronchial adenomas/carcinoids, Burkitt's lymphoma, carcinoid tumor, central nervous system lymphoma, cerebellar astrocytoma, Cervical cancer, Chronic bronchitis, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor, extragonadal germ cell tumor, extrahepatic bile duct cancer, eye cancer (intraocular melanoma, retinoblastoma), gallbladder cancer, gastric (stomach) cancer, gastrointestinal carcinoid tumor, gastrointestinal stromal tumor (GIST), germ cell tumor (extracranial, extragonadal, or ovarian), gestational trophoblastic tumor, glioma of the brain stem, gastric carcinoid, hairy cell leukemia, head and neck cancer, heart cancer, hepatocellular (liver) cancer, Hodgkin lymphoma, hypopharyngeal cancer, hypothalamic and visual pathway glioma, intraocular melanoma, islet cell carcinoma (endocrine pancreas), Kaposi sarcoma, kidney cancer (renal cell cancer), Laryngeal cancer, leukaemia (acute lymphoblastic, acute myeloid, chronic lymphocytic, chronic myelogenous), lip and oral cavity cancer, liposarcoma, liver cancer, lung cancer (non-small cell, small cell), lymphomas (AIDS-related, Burkitt, cutaneous T-Cell, Hodgkin, primary central nervous system), macroglobulinemia (Waldenström), male breast cancer, malignant fibrous histiocytoma of bone/osteosarcoma, medulloblastoma, melanoma, Merkel cell cancer, Mesothelioma, metastatic squamous neck cancer with occult primary, mouth cancer, multiple endocrine neoplasia syndrome, multiple myeloma/plasma cell neoplasm, Mycosis fungoides, myelodysplastic syndromes, myelodysplastic/myeloproliferative diseases, myelogenous leukemia, chronic, myeloid leukemia, myeloma, myeloproliferative disorders, nasal cavity and paranasal sinus cancer, nasopharyngeal carcinoma, neuroblastoma, oligodendroglioma, oral cancer, oropharyngeal cancer, osteosarcoma/malignant fibrous histiocytoma of bone, ovarian cancer, ovarian epithelial cancer, ovarian germ cell tumor, ovarian low malignant potential tumor, pancreatic cancer, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia/Multiple myeloma, pleuropulmonary blastoma, primary central nervous system lymphoma, prostate cancer, rectal cancer, renal cell carcinoma, renal pelvis and ureter, retinoblastoma, rhabdomyosarcoma, salivary gland cancer, sarcoma (Ewing family of tumors, Kaposi, soft tissue, uterine), Sezary syndrome, skin cancer (carcinoma, melanoma, non-melanoma, Merkel cell), small cell lung cancer, small intestine cancer, soft tissue sarcoma, squamous cell carcinoma, supratentorial primitive neuroectodermal tumor, testicular cancer, throat cancer, thymoma, thymoma and thymic carcinoma, thyroid cancer, transitional cell cancer of the renal pelvis and ureter, trophoblastic tumor, urethral cancer, uterine cancer, (endometrial, sarcoma), vaginal cancer, visual pathway and hypothalamic glioma, vulvar cancer, Wilms tumor (kidney cancer),

As used herein, the term “breast tumor” relates to an abnormal hyperproliferation of breast tissue cells in a subject, which may be a benign (non-cancerous) tumor or a malign (cancerous) tumor. Benign breast tumors, preferably, include fibroadenomas, granular cell tumors, intraductal papillomas, and phyllodes tumors. A malign tumor, is a breast cancer (BC) as specified herein above.

As used herein, the term “metastatic breast cancer” (MBC) relates to a breast cancer wherein cancer cells grow as a metastasis at least one secondary site, i.e. a non-adjacent organ or part of the body of a subject.

As used herein, the term “ovary tumor” relates to an abnormal hyperproliferation of ovary tissue cells in a subject, which may be a benign (non-cancerous) tumor or a malign (cancerous) tumor. A malign tumor is an ovary cancer (OvaCa) as specified herein above.

As used herein, the term “pancreatic tumor” relates to an abnormal hyperproliferation of ovary tissue cells in a subject, which may be a benign (non-cancerous) tumor or a malign (cancerous) tumor. A malign tumor is a pancreatic cancer (PaCa) as specified herein above.

The term “circulating tumor cell” or “CTC” is understood by the skilled artisan and relates to a tumor cell detached from the primary or metastatic tumor and circulating in the bloodstream. It is to be understood that the number of CTC is a prognostic marker for disease and therapy outcome in breast cancer, e.g. for overall survival. The term “CTC status” relates to the presence or absence of more than a reference amount of CTC in a sample. Preferably, the reference amount of CTC is 2, 2.5, 3, 3.5, 4, 4.5, 5, 5.5, 6, 6.5, 7, or 7.5 CTC/7.5 ml blood, 5 CTC/7.5 ml blood being more preferred. In subjects where a blood sample comprises more than said reference amount of CTC, the CTC status is unfavorable, indicating a low probability of successful treatment and a low progression-free and overall survival probability. Conversely, in subjects where a blood sample comprises less than said reference amount of CTC, the CTC status is favorable, indicating a high probability of successful treatment and a high progression-free and overall survival probability. Advantageously, it has been found in the present invention that the amounts of the miRNAs used for determining the CTC status of a subject as defined herein below are indicative of the CTC status of a subject. Thus, determining the CTC status in a subject as used herein relates to determining the amount or amounts of said miRNA or miRNAs and thus obtaining an indication of the subject's CTC status. Preferably, the status can be diagnosed to be “favorable” or “unfavorable”

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms.

The term “indicator” and “marker” are used interchangeably herein, and refer to a sign or signal for a condition or is used to monitor a condition. Such a “condition” refers to the biological status of a cell, tissue or organ or to the health and/or disease status of an individual. An indicator may be the presence or absence of a molecule, including but not limited to peptide, protein, and nucleic acid, or may be a change in the expression level or pattern of such molecule in a cell, or tissue, organ or individual. An indicator may be a sign for the onset, development or presence of a disease in an individual or for the further progression of such disease. An indicator may also be a sign for the risk of developing a disease in an individual.

As used herein, the term “gene product” relates to a, preferably macromolecular, physical entity, the presence of which in a cell depends on the expression of said gene in said cell. The mechanisms of gene expression are well-known to the one skilled in the art to include the basic mechanisms of transcription, i.e. formation of RNA corresponding to the said gene or parts thereof, and translation, i.e. production of polypeptide molecules having an amino acid sequence encoded by said RNA according to the genetic code; it is well-known to the one skilled in the art that other cellular processes may be involved in gene expression as well, e.g. RNA processing, RNA editing, proteolytic processing, protein editing, and the like. The term gene product thus includes RNA, preferably mRNA, as well as polypeptides expressed from said gene. It is clear from the above that the term gene product also includes fragments of said RNA(s), preferably with a length of at least ten, at least twelve, at least 20, at least 50, or at least 100 nucleotides, and fragments (peptides) from said polypeptides, preferably with a length of at least eight, at least ten, at least twelve, at least 15, at least 20 amino acids.

“Determining” the amount of a gene product relates to measuring the amount of said gene product, preferably semi-quantitatively or quantitatively. Measuring can be done directly or indirectly. Preferably, measuring is performed on a processed sample, said processing comprising extraction of polynucleotides or polypeptides from the sample. It is, however, also envisaged by the present invention that the gene product is determined in situ, e.g. by immunohistochemistry (IHC)

The amount of the polynucleotides of the present invention can be determined with several methods well-known in the art. Quantification preferably is absolute, i.e. relating to a specific number of polynucleotides or, more preferably, relative, i.e. measured in arbitrary normalized units. Preferably, normalization is carried out by calculating the ratio of a number of specific polynucleotides and total number of polynucleotides or a reference amplification product. Methods allowing for absolute or relative quantification are well known in the art. E.g., quantitative PCR methods are methods for relative quantification; if a calibration curve is incorporated in such an assay, the relative quantification can be used to obtain an absolute quantification. Other known methods are, e.g. nucleic acid sequence-based amplification (NASBA) or the Branched DNA Signal Amplification Assay method in combination with dot blot or luminex detection of amplified polynucleotides. Preferably, the polynucleotide amounts are normalized polynucleotide amounts, i.e. the polynucleotide amounts obtained are set into relation to at least one reference amplification product, thereby, preferably, setting the polynucleotide amounts into relation to the number of cells in the sample and/or the efficiency of polynucleotide amplification. Thus, preferably, the reference amplification product is a product obtained from a polynucleotide known to have a constant abundancy in each cell, i.e. a polynucleotide comprised in most, preferably all, cells of a sample in approximately the same amount. More preferably, the reference amplification product is amplified from a chromosomal or mitochondrial gene or from the mRNA of a housekeeping gene. The amount of polynucleotides could be determined by Shotgun sequencing, Bridge PCR, Sanger sequencing, pyrosequencing, next-generation sequencing, Single-molecule real-time sequencing, Ion Torrent sequencing, Sequencing by synthesis, Sequencing by ligation, Massively parallel signature sequencing, Polony sequencing, DNA nanoball sequencing, Heliscope single molecule sequencing, Single molecule real time (SMRT) sequencing, Nanopore DNA sequencing, Tunneling currents DNA sequencing, Sequencing by hybridization, Sequencing with mass spectrometry, Microfluidic Sanger sequencing, Transmission electron microscopy DNA sequencing, RNA polymerase sequencing, In vitro virus high-throughput sequencing, Chromatin Isolation by RNA Purification (ChIRP-Seq), Global Run-on Sequencing (GRO-Seq), Ribosome Profiling Sequencing (Ribo-Seq)/ARTseq, RNA Immunoprecipitation Sequencing (RIP-Seq), High-Throughput Sequencing of CLIP cDNA library (HITS-CLIP), Crosslinking and Immunoprecipitation Sequencing, Photoactivatable Ribonucleoside-Enhanced Crosslinking and Immunoprecipitation (PAR-CLIP), Individual Nucleotide Resolution CLIP (iCLIP), Native Elongating Transcript Sequencing (NET-Seq), Targeted Purification of Polysomal mRNA (TRAP-Seq), Crosslinking, Ligation, and Sequencing of Hybrids (CLASH-Seq), Parallel Analysis of RNA Ends Sequencing (PARE-Seq), Genome-Wide Mapping of Uncapped Transcripts (GMUCT), Transcript Isoform Sequencing (TIF-Seq), Paired-End Analysis of TSSs (PEAT), Selective 2′-Hydroxyl Acylation Analyzed by Primer Extension Sequencing (SHAPE-Seq), Parallel Analysis of RNA Structure (PARS-Seq), Fragmentation Sequencing (FRAG-Seq), CXXC Affinity Purification Sequencing (CAP-Seq), Alkaline Phosphatase Calf Intestine-Tobacco Acid Pyrophosphatase Sequencing (CIP-TAP), Inosine Chemical Erasing Sequencing (ICE), m6A-Specific Methylated RNA Immunoprecipitation Sequencing (MeRIP-Seq), Digital RNA Sequencing, Whole-Transcript Amplification for Single Cells (Quartz-Seq), Designed Primer-Based RNA Sequencing (DP-Seq), Switch Mechanism at the 5′ End of RNA Templates (Smart-Seq), Switch Mechanism at the 5′ End of RNA Templates Version 2 (Smart-Seq2), Unique Molecular Identifiers (UMI), Cell Expression by Linear Amplification Sequencing (CEL-Seq), Single-Cell Tagged Reverse Transcription Sequencing (STRT-Seq), Single-Molecule Molecular Inversion Probes (smMIP), Multiple Displacement Amplification (MDA), Multiple Annealing and Looping-Based Amplification Cycles (MALBAC), Oligonucleotide-Selective Sequencing (OS-Seq), Duplex Sequencing (Duplex-Seq), Bisulfite Sequencing (BS-Seq), Post-Bisulfite Adapter Tagging (PBAT), Tagmentation-Based Whole Genome Bisulfite Sequencing (T-WGBS), Oxidative Bisulfite Sequencing (oxBS-Seq), Tet-Assisted Bisulfite Sequencing (TAB-Seq), Methylated DNA Immunoprecipitation Sequencing (MeDIP-Seq), Methylation-Capture (MethylCap) Sequencing, Methyl-Binding-Domain-Capture (MBDCap) Sequencing, Reduced-Representation Bisulfite Sequencing (RRBS-Seq), DNase 1 Hypersensitive Sites Sequencing (DNase-Seq), MNase-Assisted Isolation of Nucleosomes Sequencing (MAINE-Seq), Chromatin Immunoprecipitation Sequencing (ChIP-Seq), Formaldehyde-Assisted Isolation of Regulatory Elements (FAIRE-Seq), Assay for Transposase-Accessible Chromatin Sequencing (ATAC-Seq), Chromatin Interaction Analysis by Paired-End Tag Sequencing (ChIA-PET), Chromatin Conformation Capture (Hi-C/3C-Seq), Circular Chromatin Conformation Capture (4-C or 4C-Seq), Chromatin Conformation Capture Carbon Copy (5-C), Retrotransposon Capture Sequencing (RC-Seq), Transposon Sequencing (Tn-Seq) or Insertion Sequencing (INSeq), Translocation-Capture Sequencing (TC-Seq), fluorescence based methods (such as: mircoarray, real-time PCR), mass-based methods (mass spectrometry), restriction enzyme based methods, antibody-immunoprecipitation based methods, and digital PCR.

The amount of peptides or polypeptides of the present invention can be determined in various ways. Direct measuring relates to measuring the amount of the peptide or polypeptide based on a signal which is obtained from the peptide or polypeptide itself and the intensity of which directly correlates with the number of molecules of the peptide present in the sample. Such a signal—sometimes referred to as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

Determining the amount of a peptide or polypeptide can be achieved by all known means for determining the amount of a peptide in a sample. Said means comprise immunoassay and/or immunohistochemistry devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, Cobalt Binding Assays, and latex agglutination assays.

Determining the amount of a peptide or polypeptide comprises the step of measuring a specific intensity signal obtainable from the peptide or polypeptide in the sample. As described above, such a signal may be the signal intensity observed at an m/z variable specific for the peptide or polypeptide observed in mass spectra or a NMR spectrum specific for the peptide or polypeptide.

As used herein, the term “CpG site” relates to a dinucleotide sequence 5′-CG-3′ comprised in a polynucleotide, preferably comprised in DNA, more preferably comprised in genomic DNA of a subject. The CpG sites to be analyzed according to the present invention are the CpG sites located in the intron, exon or promoter region of a gene of interest. In case the CpG sites are located in the promoter region, said region is preferably 3000 nucleotides, 2500 nucleotides, 2100 nucleotides, or 1750 nucleotides upstream of the translation start site of the respective gene of interest. More preferably, the CpG sites to be analyzed according to the present invention are the CpG sites located in the region 1750-3000 nucleotides, 2100-3000 nucleotides, or 2500-3000 nucleotides upstream of the translation start site of the gene of interest gene.

Thus, analysis of a CpG site corresponding to a CpG site of the present invention is also encompassed by the present invention. The skilled person knows how to determine the CpG sites in a sample corresponding to the CpG sites detailed herein above, e.g. by determining the translation start site of the gene of interest and/or by aligning said sequence from a sample to the sequence of the gene of interest. Further, it is also envisaged by the present invention that the methylation status of other CpG sites is determined in addition to determining the methylation status of a CpG site of the present invention.

The term “determining the methylation status” relates to determining if a methyl group is present at the 5 position of the pyrimidine ring of a cytosine in a polynucleotide. Preferably, the cytosine residue is followed in 3′ direction by a guanosine residue, the two residues forming a CpG site. The presence of said methyl group can be determined by various methods well known to the skilled person, including, e.g., methylation-specific PCR (MSP), whole genome bisulfite sequencing or other sequencing based methods (Bisulfite Sequencing (BS-Seq), Post-Bisulfite Adapter Tagging (PBAT), Tagmentation-Based Whole Genome Bisulfite Sequencing (T-WGBS), Oxidative Bisulfite Sequencing (oxBS-Seq), Tet-Assisted Bisulfite Sequencing (TAB-Seq), Methylated DNA Immunoprecipitation Sequencing (MeDIP-Seq), Methylation-Capture (MethylCap) Sequencing, Methyl-Binding-Domain-Capture (MBDCap) Sequencing, Reduced-Representation Bisulfite Sequencing (RRBS-Seq)), real-time PCR based methods of bisulfite treated DNA, e.g. Methylight, restriction with a methylation-sensitive restriction enzyme, e.g. in the HpaII tiny fragment enrichment by ligation-mediated PCR (HELP)-Assay, pyrosequencing of bisulfite treated DNA, or the like AIMS, amplification of inter-methylated sites; BC-seq, bisulphite conversion followed by capture and sequencing; BiMP, bisulphite methylation profiling; BS, bisulphite sequencing; BSPP, bisulphite padlock probes; CHARM, comprehensive high-throughput arrays for relative methylation; COBRA, combined bisulphite restriction analysis; DMH, differential methylation hybridization; HELP, HpaII tiny fragment enrichment by ligation-mediated PCR; MCA, methylated CpG island amplification; MCAM, MCA with microarray hybridization; MeDIP, mDIP and mCIP, methylated DNA immunoprecipitation; MIRA, methylated CpG island recovery assay; MMASS, microarray-based methylation assessment of single samples; MS-AP-PCR, methylation-sensitive arbitrarily primed PCR; MSCC, methylation-sensitive cut counting; MSP, methylation-specific PCR; MS-SNuPE, methylation-sensitive single nucleotide primer extension; NGS, next-generation sequencing; RLGS, restriction landmark genome scanning; RRBS, reduced representation bisulphite sequencing; -seq, followed by sequencing; WGSBS, whole-genome shotgun bisulphite sequencing. (Manel Esteller, Cancer epigenomics: DNA methylomes and histone-modification maps, Nature, 2007, 8:286-298; Peter W. Laird, Principles and challenges of genome-wide DNA methylation analysis. Nature Review Genetics, 2010, 11: 191-203). Preferably, the methylation status is determined by the methods described in the examples herein below, e.g. the Infinium 27K methylation assay or the mass spectrometry-based method of MALDI-TOF mass spectrometry. As such, the methylation status of a specific cytosine residue in a specific polynucleotide molecule can only be “unmethylated” (meaning 0% methylation) or “methylated” (meaning 100% methylation). In the case of a CpG site in a double-stranded DNA molecule, which comprises two cytosine residues, the methylation status can be “unmethylated” (meaning 0% methylation, i.e. none of the two cytosine residues methylated), “hemimethylated” (meaning 50% methylation, i.e. one of the two cystosine residues methylated), or “methylated” or “fully methylated” (meaning 100% methylation, i.e. both cytosine residues methylated). It is, however, understood by the person skilled in the art that if polynucleotides from a multitude of cells are obtained and the methylation status of a specific cytosine residue within said multitude of polynucleotides is determined, an average methylation status is determined, which can e.g., preferably, be expressed as a percentage (% methylation), and which can assume any value between 0% and 100%. It is also understood by the skilled person, that the methylation status can be expressed as a percentage in case the average methylation of different cell populations is determined. E.g. the blood cells according to the present invention are a mixture of variant cell types. It is possible that certain cell types have high methylation levels whereas other cell types have lower methylation levels, and finally reach an average methylation of e.g. 50%.

In some instance the methylation status of several CpG sites can be combined into a single methylation level. For example, the term GeneX_CpG.1.2.3 would refer to the mean methylation level of CpGs 1, 2 and 3 within GeneX.

As used herein, the term “detection agent” relates to an agent specifically interacting with, and thus recognizing, the expression level of a gene of interest, the methylation status of a gene of interest, or the presence or amount of a miRNA of the present invention. Preferably, said detection agent is a protein, polypeptide, peptide, polynucleotide or an oligonucleotide. Preferably, the detection agent is labelled in a way allowing detection of said detection agent by appropriate measures. Labelling can be done by various techniques well known in the art and depending of the label to be used. Preferred labels to be used are fluorescent labels comprising, inter alia, fluorochromes such as fluorescein, rhodamin, or Texas Red. However, the label may also be an enzyme or an antibody. It is envisaged that an enzyme to be used as a label will generate a detectable signal by reacting with a substrate. Suitable enzymes, substrates and techniques are well known in the art. A detection agent to be used as label may specifically recognize a target molecule which can be detected directly (e.g., a target molecule which is itself fluorescent) or indirectly (e.g., a target molecule which generates a detectable signal, such as an enzyme). The labelled detection agents of the sample will be contacted to the sample to allow specific interaction. Washing may be required to remove non-specifically bound detection agent which otherwise would yield false values. After this interaction step is complete, a researcher will place the detection device into a reader device or scanner. A device for detecting fluorescent labels, preferably, consists of some lasers, preferably a special microscope, and a camera. The fluorescent labels will be excited by the laser, and the microscope and camera work together to create a digital image of the sample. These data may be then stored in a computer, and a special program will be used, e.g., to subtract out background data. The resulting data are, preferably, normalized, and may be converted into a numeric and common unit format. The data will be analyzed to compare samples to references and to identify significant changes.

“Comparing” as used herein encompasses comparing the presence, absence or amount of an indicator referred to herein which is comprised by the sample to be analyzed with the presence, absence or amount of said indicator in a suitable reference sample. It is to be understood that comparing as used herein refers to a comparison of corresponding parameters or values, e.g., an absolute amount of the indicator as referred to herein is compared to an absolute reference amount of said indicator; a concentration of the indicator is compared to a reference concentration of said indicator; an intensity signal obtained from the indicator as referred to herein in a sample is compared to the same type of intensity signal of said indicator in a reference sample. The comparison referred to may be carried out manually or computer assisted. For a computer assisted comparison, the value of the determined amount may be compared to values corresponding to suitable references which are stored in a database by a computer program. The computer program may further evaluate the result of the comparison by means of an expert system. Accordingly, the result of the identification referred to herein may be automatically provided in a suitable output format.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis, a sample provides information about the tissue status or the health or diseased status of an organ or individual. Examples of samples include but are not limited to fluid samples such as blood, serum, plasma, synovial fluid, urine, saliva, lymphatic fluid, lacrimal fluid, and fluid obtainable from the glands such as e.g. breast or prostate, or tissue samples such as e.g. tissue extracts obtained from tumour tissue or tissue adjacent to a tumour. Further examples of samples are cell cultures or tissue cultures such as but not limited to cultures of various cancer cells.

Samples can be obtained by well-known techniques and include, preferably, scrapes, swabs or biopsies from the digestive tract, liver, pancreas, anal canal, the oral cavity, the upper aerodigestive tract and the epidermis. Such samples can be obtained by use of brushes, (cotton) swabs, spatula, rinse/wash fluids, punch biopsy devices, puncture of cavities with needles or surgical instrumentation. Tissue or organ samples may be obtained from any tissue or organ by, e.g., biopsy or other surgical procedures. More preferably, samples are samples of body fluids, e.g., preferably, blood, plasma, serum, urine, saliva, lacrimal fluid, and fluids obtainable from the breast glands, e.g. milk. Most preferably, the sample of a body fluid comprises cells of the subject. Separated cells may be obtained from the body fluids or the tissues or organs by separating techniques such as filtration, centrifugation or cell sorting. Preferably, samples are obtained from those body fluids described herein below. More preferably, cells are isolated from said body fluids as described herein below.

Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount or level.

The term “reference sample” as used herein, refers to a sample which is analysed in a substantially identical manner as the sample of interest and whose information is compared to that of the sample of interest. A reference sample thereby provides a standard allowing for the evaluation of the information obtained from the sample of interest. A reference sample may be derived from a healthy or normal tissue, organ or individual, thereby providing a standard of a healthy status of a tissue, organ or individual. Differences between the status of the normal reference sample and the status of the sample of interest may be indicative of the risk of disease development or the presence or further progression of such disease or disorder. A reference sample may be derived from an abnormal or diseased tissue, organ or individual thereby providing a standard of a diseased status of a tissue, organ or individual. Differences between the status of the abnormal reference sample and the status of the sample of interest may be indicative of a lowered risk of disease development or the absence or bettering of such disease or disorder. A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time. A reference sample was taken at an earlier or later time point in case a period of time has lapsed between taking of the reference sample and taking of the sample of interest. Such period of time may represent years (e.g. 1, 2, 3, 4, 5, 10, 15, 20, 25, 30, 35, 40, 45, 50, 60, 70, 80, 90, 100 years), months (1, 2, 3, 4, 5, 6, 7, 8, 9, 10, 11, 12 months), weeks (e.g. 1, 2, 3, 4, 5, 6, 7, 8 weeks), days (e.g. 1, 2, 3, 4, 5, 10, 15, 20, 25, 30, 35, 40, 45, 50, 60, 70, 80, 90, 100, 200, 300, 400, 500 days), hours (1, 2, 3, 4, 5, 6, 7, 8, 9, 10, 11, 12 hours), minutes (e.g. 1, 2, 3, 4, 5, 10, 15, 20, 25, 30, 35, 40, 45, 50, 60 minutes), or seconds (e.g. 1, 2, 3, 4, 5, 10, 15, 20, 25, 30, 35, 40, 45, 50, 60 seconds).

A reference sample may be “treated differently” or “exposed differently” than a sample of interest in case both samples are treated in a substantially identical way except from a single factor. Such single factors include but are not limited to the time of exposure, the concentration of exposure, or the temperature of exposure to a certain substance. Accordingly, a sample of interest may be exposed to a different dosage of a certain substance than the reference sample or may be exposed for a different time interval than the reference sample or may be exposed at a different temperature than the reference sample. Different dosages to which a sample of interest may be exposed to include but are not limited to the 2-fold, 5-fold, 10-fold, 20-fold, 30-fold, 40-fold, 50-fold, 100-fold and/or 1000-fold increased or decreased dosage of the dosage the reference sample is exposed to. Different exposure times to which a sample of interest may be exposed to include but are not limited to the 2-fold, 5-fold, 10-fold, 20-fold, 30-fold, 40-fold, 50-fold, 100-fold and/or 1000-fold longer or shorter time period than the exposure of the reference. Different temperatures of exposure to which a sample of interest may be exposed to include but are not limited to the 2-fold, 5-fold, 10-fold, 20-fold, 30-fold, 40-fold, 50-fold, 100-fold and/or 1000-fold increased or decreased temperature than the exposure of the reference. In a non-limiting example a sample of interest may be exposed to a 10-fold increased concentration of a substance than the reference sample. The analysis of both samples is then conducted in a substantially identical manner allowing determining the effects, i.e. a beneficial or an adverse effect, of the increased concentration of such substance on the sample of interest. The skilled person will appreciate that this example applies mutatis mutandis to different ranges of concentrations, different exposure times, and/or different temperatures at exposure.

The terms “lowered” or “decreased” level of an indicator refer to the level of such indicator in the sample being reduced in comparison to the reference or reference sample. The terms “elevated” or “increased” level of an indicator refers to the level of such indicator in the sample being higher in comparison to the reference or reference sample.

Reference amounts can, in principle, be calculated for a group or cohort of subjects as specified herein based on the average or median values for a given miRNA by applying standard methods of statistics. In particular, accuracy of a test such as a method aiming to diagnose an event, or not, is best described by its receiver-operating characteristics (ROC) (see especially Zweig 1993, Clin. Chem. 39:561-577). The ROC graph is a plot of all of the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1-specificity for the complete range of thresholds suitable for making a distinction. On the y-axis is sensitivity, or the true-positive fraction, which is defined as the ratio of number of true-positive test results to the sum of number of true-positive and number of false-negative test results. This has also been referred to as positivity in the presence of a disease or condition. It is calculated solely from the affected subgroup. On the x-axis is the false-positive fraction, or 1-specificity, which is defined as the ratio of number of false-positive results to the sum of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 450 diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis or prediction for a given event with a proper balance of sensitivity and specificity, respectively. Accordingly, the reference to be used for the methods of the present invention can be generated, preferably, by establishing a ROC for said cohort as described above and deriving a threshold amount there from. Dependent on a desired sensitivity and specificity for a diagnostic method, the ROC plot allows deriving suitable thresholds. Preferably, the reference amounts lie within the range of values that represent a sensitivity of at least 75% and a specificity of at least 45%, or a sensitivity of at least 80% and a specificity of at least 40%, or a sensitivity of at least 85% and a specificity of at least 33%, or a sensitivity of at least 90% and a specificity of at least 25%.

Preferably, the reference amount as used herein is derived from samples of subjects obtained before treatment, but for which it is known if their donors were being afflicted with BC or MBC or not. This reference amount level may be a discrete figure or may be a range of figures. Evidently, the reference level or amount may vary between individual species of miRNA. The measuring system therefore, preferably, is calibrated with a sample or with a series of samples comprising known amounts of each specific miRNA. It is understood by the skilled person that in such case the amount of miRNA can preferably be expressed as arbitrary units (AU). Thus, preferably, the amounts of miRNA are determined by comparing the signal obtained from the sample to signals comprised in a calibration curve. The reference amount applicable for an individual subject may vary depending on various physiological parameters such as age or subpopulation. Thus, a suitable reference amount may be determined by the methods of the present invention from a reference sample to be analyzed together, i.e. simultaneously or subsequently, with the test sample. Moreover, a threshold amount can be preferably used as a reference amount. A reference amount may, preferably, be derived from a sample of a subject or group of subjects being afflicted with BC or MBC which is/are known to be afflicted with BC or MBC. A reference amount may, preferably, also be derived from a sample of a subject or group of subjects known to be not afflicted with BC or MBC. It is to be understood that the aforementioned amounts may vary due to statistics and errors of measurement. A deviation, i.e. a decrease or an increase of the miRNA amounts referred to herein is, preferably, a statistically significant deviation, i.e. a statistically significant decrease or a statistically significant increase.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in an individual that have previously had the disorder(s); and (e) limiting or preventing recurrence of symptoms in individuals that were previously symptomatic for the disorder(s).

As used herein, “prevent”, “preventing”, “prevention”, or “prophylaxis” of a disease or disorder means preventing that such disease or disorder occurs in patient.

As used herein, the term “therapy” refers to all measures applied to a subject to ameliorate the diseases or disorders referred to herein or the symptoms accompanied therewith to a significant extent. Said therapy as used herein also includes measures leading to an entire restoration of the health with respect to the diseases or disorders referred to herein. It is to be understood that therapy as used in accordance with the present invention may not be effective in all subjects to be treated. However, the term shall require that a statistically significant portion of subjects being afflicted with a disease or disorder referred to herein can be successfully treated. Whether a portion is statistically significant can be determined without further ado by the person skilled in the art using various well-known statistic evaluation tools discussed herein above.

The term “breast cancer therapy”, as used herein, relates to applying to a subject afflicted with breast cancer, including metastasizing breast cancer, measures to remove cancer cells from the subject, to inhibit growth of cancer cells, to kill cancer cells, or to cause the body of a patient to inhibit the growth of or to kill cancer cells. Preferably, breast cancer therapy is chemotherapy, anti-hormone therapy, targeted therapy, immunotherapy, or any combination thereof. It is, however, also envisaged that the cancer therapy is radiation therapy or surgery, alone or combination with other therapy regimens. It is understood by the skilled person that the selection of the breast cancer therapy depends on several factors, like age of the subject, tumor staging, and receptor status of tumor cells. It is, however, also understood by the person skilled in the art, that the selection of the breast cancer therapy can be assisted by the methods of the present invention: if, e.g. BC is diagnosed by the method for diagnosing BC, but no MBC is diagnosed by the method for diagnosing MBC, surgical removal of tumor may be sufficient. If, e.g. BC is diagnosed by the method for diagnosing BC and MBC is diagnosed by the method for diagnosing MBC, therapy measures in addition to surgery, e.g. chemotherapy and/or targeted therapy, may be appropriate. Likewise, if, e.g. BC is diagnosed by the method for diagnosing BC, and an unfavorable CTC status is determined by the method for determining the CTC status, e.g. a further addition of immunotherapy to the therapy regimen may be required.

As used herein, the term “chemotherapy” relates to treatment of a subject with an antineoplastic drug. Preferably, chemotherapy is a treatment including alkylating agents (e.g. cyclophosphamide), platinum (e.g. carboplatin), anthracyclines (e.g. doxorubicin, epirubicin, idarubicin, or daunorubicin) and topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)), antiangiogenic agents (e.g. Bevacizumab), or Iodine131-1-(3-iodobenzyl)guanidine (therapeutic metaiodobenzylguanidine), histone deacetylase (HDAC) inhibitors, alone or any suitable combination thereof. It is to be understood that chemotherapy, preferably, relates to a complete cycle of treatment, i.e. a series of several (e.g. four, six, or eight) doses of antineoplastic drug or drugs applied to a subject separated by several days or weeks without such application.

The term “anti-hormone therapy” relates to breast cancer therapy by blocking hormone receptors, e.g. estrogen receptor or progesterone receptor, expressed on tumor cells, or by blocking the biosynthesis of estrogen. Blocking of hormone receptors can preferably be achieved by administering compounds, e.g. tamoxifen, binding specifically and thereby blocking the activity of said hormone receptors. Blocking of estrogen biosynthesis is preferably achieved by administration of aromatase inhibitors like, e.g. anastrozole or letrozole. It is known to the skilled artisan that anti-hormone therapy is only advisable in cases where tumor cells are expressing hormone receptors.

The term “targeted therapy”, as used herein, relates to application to a patient a chemical substance known to block growth of cancer cells by interfering with specific molecules known to be necessary for tumorigenesis or cancer or cancer cell growth. Examples known to the skilled artisan are small molecules like, e.g. PARP-inhibitors (e.g. Iniparib), or monoclonal antibodies like, e.g., Trastuzumab.

The term “immunotherapy” as used herein relates to the treatment of cancer by modulation of the immune response of a subject. Said modulation may be inducing, enhancing, or suppressing said immune response. The term “cell based immunotherapy” relates to a breast cancer therapy comprising application of immune cells, e.g. T-cells, preferably tumor-specific NK cells, to a subject.

The terms “radiation therapy” or “radiotherapy” is known to the skilled artisan. The term relates to the use of ionizing radiation to treat or control cancer. The skilled person also knows the term “surgery”, relating to operative measures for treating breast cancer, e.g. excision of tumor tissue.

As used herein, the term “therapy monitoring” relates to obtaining an indication on the effect of a treatment against cancer on the cancer status of a subject afflicted with said cancer. Preferably, therapy monitoring comprises application of a method of the present invention on two samples from the same subject, wherein a first sample is obtained at a time point before the second sample. Preferably, the time point of obtaining the first sample is separated from the time point of obtaining the second sample by about one week, about two weeks, about three weeks, about for weeks, about five weeks, about, six weeks, about seven weeks, about two months, about three months, about five months, about six month, or more than about six months. It is, however, also envisaged by the present invention that the method of therapy monitoring is used for long-term monitoring of subjects, e.g. monitoring the time of relapse-free survival or the like. In such case, the time point of obtaining the first sample is separated from the time point of obtaining the second sample, preferably, by at least six months, at least one year, at least two years, at least three years, at least four years, at least five years, or at least six years. It is known to the person skilled in the art that the first sample is preferably obtained before cancer therapy is started, while the second sample is preferably obtained after therapy is started. It is, however, also envisaged by the present invention that both samples are obtained after therapy is started. The skilled artisan also understands that more than two successive samples may be obtained according to the method for therapy monitoring of the present invention and that in such case the sample obtained at the first point in time may be used as the first sample relative to the second sample as well as for a third sample. Mutatis mutandis, the sample obtained at the second point in time may nonetheless be used as a first sample relative to a third sample, and the like.

The term “treatment success”, as used herein, preferably relates to an amelioration of the diseases or disorders referred to herein or the symptoms accompanied therewith to a significant extent. More preferably, the term relates to a complete cure of said subject, i.e. to the prevention of progression and/or relapse of metastasizing breast cancer for at least five years. Accordingly, “determining treatment success” relates to assessing the probability according to which a subject was successfully treated. Preferably, the term relates to predicting progression free survival and/or overall survival of the subject, more preferably for a specific period of time. The term “predicting progression free survival” relates to determining the probability of a subject surviving without relapse and/or progression of metastatic breast cancer for a specific period of time. Accordingly, the term “predicting overall survival” relates to determining the probability according to which a subject will survive for a specific period of time. Preferably, said period of time is at least 12 months, more preferably at least 24 months.

The terms “pharmaceutical”, “medicament” and “drug” are used interchangeably herein referring to a substance and/or a combination of substances being used for the identification, prevention or treatment of a tissue status or disease.

The term “kit” as used herein refers to a collection of the aforementioned components, preferably, provided separately or within a single container. The container, also preferably, comprises instructions for carrying out the method of the present invention. Examples for such the components of the kit as well as methods for their use have been given in this specification. The kit, preferably, contains the aforementioned components in a ready-to-use formulation. Preferably, the kit may additionally comprise instructions, e.g., a user's manual for adjusting the components, e.g. concentrations of the detection agents, and for interpreting the results of any determination(s) with respect to the diagnoses provided by the methods of the present invention. Particularly, such manual may include information for allocating the amounts of the determined a gene product to the kind of diagnosis. Details are to be found elsewhere in this specification. Additionally, such user's manual may provide instructions about correctly using the components of the kit for determining the amount(s) of the respective biomarker. A user's manual may be provided in paper or electronic form, e.g., stored on CD or CD ROM. The present invention also relates to the use of said kit in any of the methods according to the present invention.

The terms “cT”, “cN”, “cM”, “pT”, “pN” and “pM” refer to the Union for international cancer control (UICC) classification of malignant tumors (TNM). The three main parameters T, N and M describe the primary tumor site (T), regional lymph node involvement (N) and distant metastatic spread (M). These parameters can be combined with prefixes such as “c” which would indicate that the stage is determined from evidence acquired clinically before treatment (e.g. examination, laboratory tests, imaging or biopsy). The prefix “p” would indicate the results of detailed post-surgical pathologic TNM classification.

The term “Qubit” refers to a type of fluorescent-based method able to accurately quantify the concentration of nucleic acids in a given sample. Qubit provides specific fluorescent dyes to quantify DNA, RNA, miRNA or proteins. These dyes have extremely low fluorescence until bound to their target molecule, thus giving specificity and accuracy to the quantification of nucleic acids. Qubit preferably refers to levels of cell-free miRNA.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

### EMBODIMENTS

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a method of diagnosing or prognosing cancer in a subject, comprising the steps of determining in vitro in a sample obtained from said subject
a) the cytosine methylation of at least one CpG dinucleotide within at least one gene selected from the group consisting of HYAL2, MGRN1, RPTOR, SLC22A18, FUT7, RAPSN and S100P and/or
b) the expression level of at least one miRNA selected from the group consisting of miR-148b, miR-409, miR-652, miR-200c, miR-375, miR-320b and miR-141 with the proviso that the at least one miRNA comprises at least one miRNA selected from the group consisting of miR-200c-3p, miR-375 and miR-320b,
   wherein the method optionally further comprises determining the expression level of miR-451a, wherein a decreased level of cytosine methylation of at least one CpG dinucleotide within the at least one gene and an altered expression level of the at least one miRNA is indicative of the present and/or future cancer disease state in said subject.

In other words, the claimed method uses the level of DNA methylation of a selection of biomarkers and/or the expression level of a selection of miRNAs in a sample derived from a subject to detect or predict cancer in said subject. All of the above CpG dinucleotides and miRNAs can be used as univariate markers or as multivariate markers.

In a preferred embodiment of the first aspect of the present invention, the term miR-148b refers to the sequence of the -3p or 5-p strand (preferably the -3p strand), the term miR-409 refers to the sequence of the -3p or -5p strand (preferably the -3p strand), the term miR-652 refers to the sequence of the -3p or -5p strand (preferably the -3p strand), the term miR-200c refers to the sequence of the -3p or -5p strand (preferably the -3p strand), the term miR-375 refers to the sequence of the -3p or -5p strand (preferably the -3p strand) and the term miR-320b refers to the sequence of the -3p or -5p strand (preferably the -3p strand).

In a preferred embodiment of the first aspect of the present invention, an alteration in the methylation status of HYAL2, MGRN1, RPTOR, SLC22A18, FUT7, RAPSN and/or S100P, indicates a change in tissue status or cancer disease status such as the worsening or bettering of a tissue status or cancer disease status. In particular, a decreased level of cytosine methylation of at least one CpG dinucleotide within the at least one gene selected from the group consisting of HYAL2, MGRN1, RPTOR, SLC22A18, FUT7, RAPSN and S100P is indicative of the presence of cancer. In particular, a decreased level of cytosine methylation of at least one CpG dinucleotide within the at least one gene selected from the group consisting of HYAL2, MGRN1, RPTOR, SLC22A18, FUT7, RAPSN and S100P is indicative of the increased likelihood of developing cancer. In particular, a decreased level of cytosine methylation of at least one CpG dinucleotide within the at least one gene selected from the group consisting of HYAL2, MGRN1, RPTOR, SLC22A18, FUT7, RAPSN and S100P is indicative of the presence of cancer and is indicative of the increased likelihood of developing cancer. An increased likelihood of developing cancer is used in the meaning of developing de novo cancer or the developing of new tumours.

In a preferred embodiment of the first aspect of the invention an alteration in the miR-expression level of miR-148b, miR-409, miR-652, miR-200c, miR-375, miR-320b and/or miR-141 indicates a change in tissue status or disease such as the worsening or bettering of a tissue status or disease, in particular cancer.

In a preferred embodiment of the first aspect of the present invention, the miRNAs of step b) are selected from miR-200c, miR-375, miR-148b, miR-409 and miR-652, preferably for diagnosis of cancer. More preferred the miRNAs are selected from or are miR-200c and miR-375, preferably for diagnosis of cancer, preferably ovarian cancer. More preferred the miRNAs are selected from or are miR-148b, miR-375, miR-409 and miR-652, preferably for diagnosis of cancer, preferably breast cancer.

In a preferred embodiment of the first aspect of the invention the genes of step a) are selected from HYAL2, SLC22A18, RAPSN and FUT7, preferably for diagnosis of cancer. More preferred the genes are selected from or are HYAL2 and SLC22A18, preferably for diagnosis of cancer, preferably ovarian cancer. More preferred the genes are selected from or are RAPSN and FUT7, preferably for the diagnosis of cancer, preferably breast cancer. The genes indicated as preferred genes can be combined with the miRNAs indicated as preferred miRNAs for the same purpose. Examples of these combinations are miRNAs selected from are being miR-200c and miR-375 together with genes selected from or being HYAL2 and SLC22A18, preferably for the diagnosis of cancer, preferably ovarian cancer. Another example are the miRNAs selected from or being miR-148b, miR-375, miR-409 and miR-652 in combination with the genes selected from or being RAPSN and FUT7, preferably for the diagnosis of cancer, preferably breast cancer.

In a preferred embodiment of the first aspect of the invention the miRNAs of step b) are selected from miR-375, miR-652, miR-200c, miR-320b, miR-141, preferably for prognosis of cancer. More preferred the miRNAs are selected from or are miR-375, miR-652 and miR-200c, even more preferred miR-375, preferably for prognosis of cancer, preferably ovarian cancer.

More preferred the miRNAs are selected from or are miR-200c, miR-320b, miR-141, even more preferred miR-200c, preferably for prognosis of cancer, preferably breast cancer.

In a preferred embodiment of the first aspect of the invention the genes of step a) are selected from the genes of step a) are selected from HYAL2, S100P, FUT7, SLC22A18, MGRN1 and RPTOR, preferably for prognosis of cancer. More preferred the genes are selected from or are HYAL2, S100P, FUT7, SLC22A18 and MGRN1, even more preferred HYAL2 and S100P, preferably for prognosis of cancer, preferably ovarian cancer. More preferred the genes are selected from or are HYAL2, S100P and RPTOR, preferably for prognosis of cancer, preferably breast cancer. The genes indicated as preferred genes can be combined with the miRNAs indicated as preferred miRNAs for the same purpose. Examples of these combinations are miRNAs selected from or being miR-652 and miR-200c in combination with the genes selected from or being HYAL2, S100P, FUT7, SLC122A18 and MGRN1, preferably for the prognosis of cancer, preferably ovarian cancer. Another example is miRNA miR-375 in combination with genes selected from or being HYAL2 and S100P, preferably for the prognosis of cancer, preferably ovarian cancer. Yet another example are miRNAs selected from or being miR-200c, miR-320b and miR-141 in combination with genes selected from or being HYAL2, S100P and RPTOR. Yet another example are miRNAs selected from or being miR-200c, miR-320b and miR-375 in combination with genes selected from or being HYAL2, S100P and RPTOR.

In a preferred embodiment of the first aspect of the present invention, the methylation level of at least one CpG selected from HYAL2_CpG4, S100P_CpG4; SLC22A18 CpG3, RPTOR_CpG2, RPASN_CpG5; MGRN1_CpG12 and FUT7_CpG7 is determined in step a) and the expression level of at least one of the following miRNAs is determined in step b) miR-148b, miR-409, preferably -409-3p, miR-652, preferably miR-652-3p, miR-200c, preferably -200c-3p, miR-375, miR-320b and optionally miR-451a. In an alternative embodiment S100P_CpG7 is determined instead of or additionally to S100P_CpG4.

In a preferred embodiment of the first aspect of the present invention, the methylation level of at least two CpGs selected from HYAL2_CpG4, S100P_CpG4; SLC22A18 CpG3, RPTOR_CpG2, RPASN_CpG5; MGRN1_CpG12 and FUT7_CpG7 is determined in step a) and the expression level of at least two of the following miRNAs is determined in step b) miR-148b, miR-409, preferably -409-3p, miR-652, preferably miR-652-3p, miR-200c, preferably -200c-3p, miR-375, miR-320b and optionally miR-451a. In an alternative embodiment S100P_CpG7 is determined instead of or additionally to S100P_CpG4.

In a preferred embodiment of the first aspect of the present invention, the methylation level of HYAL2_CpG4, S100P_CpG4; SLC22A18 CpG3, RPTOR_CpG2, RPASN_CpG5; MGRN1_CpG12 and FUT7_CpG7 is determined in step a) and the expression level of the following miRNAs is determined in step b) miR-148b, miR-409, preferably -409-3p, miR-652, preferably miR-652-3p, miR-200c, preferably -200c-3p, miR-375, miR-320b and optionally miR-451a. In an alternative embodiment S100P_CpG7 is determined instead of or additionally to S100P_CpG4.

In a preferred embodiment of the first aspect of the invention in addition to the cytosine methylation of step a) and/or the miRNA expression level of step b) at least one clinical marker is determined, preferably selected from Age of patient, CA125, Qubit, disease Burden, Breast Cancer Type, Stage, cT, cN, cM, “pT (Surgery)”, “pN (Surgery)”, pM, chemotherapy (yes or no) Nationality, Height [cm], Weight [kg], Age at first period, High Cholesterol, Diabetes, Endometriosis, Myom, Ovarian cyst, PCO, Autoimmune disease, Medication, Pregnancies, Age at first pregnancy, Contraceptives or hormones, Smoker, Vegetarian and Sports. Most preferred clinical markers are Age, CA125, cT, cN, cM, “pT (Surgery)”, “pN (Surgery)”, pM and Qubit.

In a preferred embodiment of the first aspect of the invention in addition to the cytosine methylation of step a) and/or the miRNA expression level of step b) the clinical marker age of patient is determined.

In a preferred embodiment of the first aspect of the invention in addition to the cytosine methylation of step a) and/or the miRNA expression level of step b) the clinical marker CA125 is determined.

In a preferred embodiment of the first aspect, an alteration in the expression level of miR-148b, miR-409, miR-652, miR-200c, miR-375 and/or miR-320b, indicates a change in tissue status or cancer disease status such as the worsening or bettering of a tissue status or cancer disease status. In particular, an increase of the expression level of a miRNA selected from miR-148b, miR-409-3p, miR-652-3p, miR-200c-3p, miR-320b is indicative of the presence of cancer and/or increased likelihood of developing cancer. A decrease of the expression level of miR-375 is indicative of the presence of cancer and/or increased likelihood of developing cancer.

In a preferred embodiment of the first aspect of the present invention, the expression level of at least one gene selected from the group consisting of HYAL2, MGRN1, RPTOR, SLC22A18, FUT7, RAPSN and S100P is determined in addition to the cytosine methylation determined in step a), wherein an increased expression level is indicative of the present and/or future cancer disease state. An increased expression level is indicative of the presence of cancer and/or increased likelihood of developing cancer.

In a preferred embodiment of the first aspect of the present invention, the expression level of at least one gene selected from the group consisting of HYAL2, MGRN1, RPTOR, SLC22A18, FUT7, RAPSN and S100P is determined alternatively to the cytosine methylation determined in step a), wherein an increased expression level is indicative of the present and/or future cancer disease state. An increased expression level is indicative of the presence of cancer and/or increased likelihood of developing cancer.

In preferred embodiments, the determination of the methylation status comprises determining methylation of at least one CpG site within the HYAL2, MGRN1, RPTOR, SLC22A18, FUT7, RAPSN and/or S100P gene. In particular, the methylation status of the promoter, intron and/or exon region of said genes is determined.

In particular, the HYAL2 gene is the human HYAL2 gene located on human chromosome 3 (Genbank Acc No: NC_000003.11 GI: 224589815). In particular, the methylation status of at least one of the CpG sites located between position 50334760 and position 50335700 on human chromosome 3 is determined. More specifically, in particular referring to build 36.1/hg18 of the human genome, the methylation status of at least one of the CpG sites located at position 50335694 (cg27091787), 50335584 (HYAL_CpG_1), 50335646 (HYAL_CpG_2), or 50335671 (HYAL_CpG_3), 50335166 (HYAL-is-310 CpG_1), 50335180 (HYAL-is-310 CpG_2), 50335192 (HYAL-is-310 CpG_3), 50335195 (HYAL-is-310 CpG_4), 50335227 (HYAL-is-310 CpG_5), 50335233 (HYAL-is-310 CpG_6), 50335300 (HYAL-is-310 CpG_7), 50335315 (HYAL-is-310 CpG_8), 50335375 (HYAL-is-310 CpG_9), 50335392 (HYAL-is-310 CpG_10), 50335401 (HYAL-is-310 CpG_11), 50334744 (HYAL2-is-325_CpG_1), 50334761 (HYAL2-is-325_CpG_2), 50334804 (HYAL2-is-325_CpG_3), 50334844 (HYAL2-is-325_CpG_4), 50334853 (HYAL2-is-325_CpG_5), 50334862 (HYAL2-is-325_CpG_6), 50334880 (HYAL2-is-325_CpG_7), 50334906 (HYAL2-is-325_CpG_8), 50334913 (HYAL2-is-325_CpG_9), 50334917 (HYAL2-is-325_CpG_10), 0334928 (HYAL2-is-325_CpG_11), 50334944 (HYAL2-is-325_CpG_12), 50334956 (HYAL2-is-325_CpG_13), 50334980 (HYAL2-is-325_CpG_14), 50334982 (HYAL2-is-325_CpG_15), 50335010 (HYAL2-is-325_CpG_16) 50335014 (HYAL2-is-325_CpG_17), 50331237 (cg08776109) and 50330420 (cg06721473) is determined.

Most specifically, at least one CpG site is selected from the list consisting of HYAL2_CpG_2 at position 50335646, and HYAL2_CpG_3 at position 50335671 and HYAL2_CpG_4 at position 50335195. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the HYAL2 promoter region comprised in the sample to be analyzed e.g the HYAL2 gene is located on Chromosome 3: positions 50,355,221-50,360,337 in build37/hg19, but on Chromosome 3: positions 50,330,244-50,335,146 in build36/hg18.

In a preferred embodiment, all CpGs within 100 bp, 80 bp, 70 bp, 60 bp, 50 bp, 40 bp, 30 bp, 20 bp, 10 bp, 5 bp distance of herein defined CpGs are measured to yield a mean methylation value of the indicated region. This embodiment is based on the phenomenon of co-methylation of neighbouring CpGs that is known particular in cancer patients. Most common is co-methylation in the areas of CpG islands and/or CpG shores.

In particular, the MGRN1 gene is the human MGRN1 gene located at human chromosome 16 (Genbank Acc No: NC_000016.10, range: 4624824-4690974, Reference GRCh38 Primary Assembly; Genbank Acc No: NC_018927.2, range: 4674882-4741756, alternate assembly CHM1_1.1; Genbank Acc No: AC_000148.1, range: 4641815-4707494, alternate assembly HuRef). In particular, the methylation status of at least one of the CpG sites located between position 4654000 and position 4681000 on human chromosome 16 is determined. In particular, the CpG site(s) is/are located in one or more of the following regions of chromosome 16: 4670069-4670542, 4654000-4655000, 4669000-4674000, and 4678000-4681000. More specifically, in particular referring to build 36.1/hg18 of the human genome, the methylation status of at least one of the CpG sites located at position: 4670487 (MGRN1_CpG_1), 4670481 (MGRN1_CpG_2), 4670466 (MGRN1_CpG_3), 4670459 (MGRN1_CpG_4), 4670442 (MGRN1_CpG_5), 4670440 (MGRN1_CpG_6), 4670435 (MGRN1_CpG_7), 4670433 (MGRN1_CpG_8), 4670422 (MGRN1_CpG_9), 4670414 (MGRN1_CpG_10), 4670411 (MGRN1_CpG_11), 4670402 (MGRN1_CpG_12), 4670393 (MGRN1_CpG_13), 4670357 (MGRN1_CpG_14), 4670352 (MGRN1_CpG_15), 4670343 (MGRN1_CpG_16), 4670341 (MGRN1_CpG_17), 4670336 (MGRN1_CpG_18), 4670313 (MGRN1_CpG_19), 4670310 (MGRN1_CpG_20), 4670301 (MGRN1_CpG_21), 4670292 (MGRN1_CpG_22), 4670287 (MGRN1_CpG_23), 4670281 (MGRN1_CpG_24), 4670276 (MGRN1_CpG_25), 4670264 (MGRN1_CpG_26), 4670234 (MGRN1_CpG_27), 4670211 (MGRN1_CpG_28), 4670180 (MGRN1_CpG_29), 4670174 (MGRN1_CpG_30), 4670157 (MGRN1_CpG_31), 4670137 (MGRN1_CpG_32), 4670123 (MGRN1_CpG_33), 4670117 (MGRN1_CpG_34). Most specifically, at least one CpG site is selected from the list consisting of MGRN1_CpG_2 at position 4670481, MGRN1_CpG_4 at position 4670459, MGRN1_CpG_12 at position 4670402 and MGRN1_CpG_26 at position 4670264. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the MGRN1 promoter region comprised in the sample to be analyzed.

In particular, the RPTOR gene is the human RPTOR gene located at human chromosome 17 (Genbank Acc No: NC_000017.11, range: 80544825-80966373, GRCh38 Primary Assembly; Genbank Acc No: NG_013034.1, range: 5001-426549, RefSeqGene; Genbank Acc No: NC_018928.2, range: 78604958-79026514, Alternate assembly CHM1_1.1; Genbank Acc No: NG_013034.1; Genbank Acc No: AC_000149.1, range: 73954508-74378467, alternate assembly HuRef). In particular, the methylation status of at least one of the CpG sites located between position 76.297.000 and position 76.416.000 on human chromosome 17 is determined. In particular, the CpG site(s) is/are located in one or more of the following regions of chromosome 17: 76.369.937-76.370.536, 76.297.000-76.310.000, 76.333.000-76.341.000, 76.360.000-76.380.000, and 76.411.000-76.416.000. More specifically, in particular referring to build 36.1/hg18 of the human genome, the methylation status of at least one of the CpG sites located at position: 76370001 (RPTOR_CpG_1), 76370037 (RPTOR_CpG_2), 76370073 (RPTOR_CpG_3), 76370092 (RPTOR_CpG_4), 76370172 (RPTOR_CpG_5), 76370199 (RPTOR_CpG_6), 76370220 (RPTOR_CpG_7), 76370253 (RPTOR_CpG_8). Most specifically, at least one CpG site is selected from the list consisting of RPTOR_CpG_2 at position 76370037, RPTOR_CpG_5 at position 76370172 and RPTOR_CpG_8 at position 76370253. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the RPTOR promoter region comprised in the sample to be analyzed.

In particular, the SLC22A18 gene is the human SLC22A18 gene located at human chromosome 11 (Genbank Acc No: NC_000011.10, range: 2899721-2925246, Reference GRCh38 primary assembly; Genbank Acc No: NG_011512.1, range: 5001-30526, RefSeqGene; Genbank Acc No: NT_187585.1, range: 131932-157362, Reference GRCh38 ALT_REF_LOCI_1; Genbank Acc No: AC_000143.1, range: 2709509-2734907, alternate assembly HuRef, Genbank Acc No: NC_018922.2, range: 2919878-2945340, alternate assembly CHM1_1.1). In particular, the methylation status of at least one of the CpG sites located between position 2876000 and position 2883000 on human chromosome 11 is determined. In particular the CpG sites are located at 2.877.113-2.877.442. More specifically, chr11: 2.876.000-chr11: 2.883.000, a 7000 bp cancer-associated, in particular BC, OvaCa, and/or PaCA-associated, differential methylation region covering, the promoter region, a CpG island and part of the gene body region of SLC22A18 (transcript variants). More specifically, in particular referring to build 36.1/hg18 of the human genome, the methylation status of at least one of the CpG sites located at position: 2877395 (SLC22A18_CpG_1), 2877375 (SLC22A18_CpG_2), 2877365 (SLC22A18_CpG_3), 2877341 (SLC22A18_CpG_4), 2877323 (SLC22A18_CpG_5), 2877311 (SLC22A18_CpG_6), 2877193 (SLC22A18_CpG_7), 2877140 (SLC22A18_CpG_8). Most specifically, at least one CpG site is selected from the list consisting of SLC22A18_CpG_3 at position 2877365, SLC22A18_CpG_4 at position 2877341 and SLC22A18_CpG_8 at position 2877140. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the SLC22A18 promoter region comprised in the sample to be analyzed.

In particular, the FUT7 gene is the human FUT7 gene located at human chromosome 9 (Genbank Acc No: NC_000009.12, range: 137030174-137032840, Reference GRCh38 primary assembly; Genbank Acc No: NG_007527.1, range: 5001-7667, RefSeqGene; Genbank Acc No: AC_000141.1, range: 109383478-109386144, Alternate assembly HuRef; Genbank Acc No: NC_018920.2, range: 140073389-140076055, Alternate assembly CHM1_1.1). In particular, the methylation status of at least one of the CpG sites located between position 139046000 and position 139048000 on human chromosome 9 is determined. More specifically, a 2000 bp BC, OvaCa, and/or PaCA-associated differential methylation region located at the promoter region of FUT7. In particular the CpG sites are located at 139.047.218-139.047.610, 139.046.000-139.048.000, and 139.045.065-139.045.817. More specifically, in particular referring to build 36.1/hg18 of the human genome, the methylation status of at least one of the CpG sites located at position: 139047253 (FUT_CpG_1), 139047314 (FUT_CpG_2), 139047346 (FUT_CpG_3), 139047427 (FUT_CpG_4), 139047445 (FUT_CpG_5), 139047467 (FUT_CpG_6), 139047483 (FUT_CpG_7), 139047566 (FUT_CpG_8). Most specifically, at least one CpG site is selected from FUT7_CpG_3 at position 139047346 and FUT7_CpG_7 at position 139047483. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the FUT7 promoter region comprised in the sample to be analyzed.

In particular, the RAPSN gene is the human RAPSN gene located at human chromosome 11 (Genbank Acc No: NC_000011.10, range: 47437757-0.47449178, Reference GRCh38 primary assembly; Genbank Acc No: NG_008312.1, range: 5001-16423, RefSeqGene; Genbank Acc No: NC_018922.2, range: 47458570-47469991, alternate assembly CHM1_1.1; Genbank Acc No: AC_000143.1, range: 47159075-47170494, alternate assembly HuRef). In particular, the methylation status of at least one of the CpG sites located between position 47427500 and position 47428500 on human chromosome 11 is determined. Preferably the CpG sites are located at 47427500-47428300. More specifically, a 1000 bp cancer-associated, preferably BC, OvaCa, and/or PaCA-associated, differential methylation region located at the promoter region of RAPSN. More specifically, in particular referring to build 36.1/hg18 of the human genome, the methylation status of at least one of the CpG sites located at position: 47427787 (RAPSN_CpG_1), 47427825 (RAPSN_CpG_2), 47427883 (RAPSN_CpG_3), 47427915 (RAPSN_CpG_4), 47427930 (RAPSN_CpG_5), 47427976 (RAPSN_CpG_6), 47428029 (RAPSN_CpG_7), 47428110 (RAPSN_CpG_8). Most specifically, at least one CpG site is selected from RAPSN_CpG_2 at position 47427825, RAPSN_CpG_4 at position 47427915, RAPSN_CpG_5 at position 47427930, RAPSN_CpG_7 at position 47428029 and RAPSN_CpG_8 at position 47428110. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the RAPSN promoter region comprised in the sample to be analyzed.

In particular, the S100P gene is the human S100P gene located at human chromosome 4 (Genbank Acc No: NC_000004.12, range: 6693839-6697170, Reference GRCh38 primary assembly; Genbank Acc No: AC_000136.1, range: 6627254-6630595, alternate assembly HuRef, Genbank Acc No: NC_018915.2, range: 6693944-6697285, alternate assembly CHM1_1.1). In particular, the methylation status of at least one of the CpG sites located between position 6746000 and position 6747000 on human chromosome 4 is determined. More specifically, a 1000 bp cancer-associated (preferably BC, OvaCa, and/or PaCA-associated) differential methylation region located from the promoter region till the first exon of S100P. In particular the CpG sites are located at 6.746.537-6.746.823. More specifically, in particular referring to build 36.1/hg18 of the human genome, the methylation status of at least one of the CpG sites located at position: 6746565 (S100P_CpG_1), 6746599 (S100P_CpG_2), 6746609 (S100P_CpG_3), 6746616 (S100P_CpG_4), 6746623 (S100P_CpG_5), 6746634 (S100P_CpG_6), 6746710 (S100P_CpG_7), 6746728 (S100P_CpG_8), 6746753 (S100P_CpG_9), 6746779 (S100P_CpG_10), 6746788 (S100P_CpG_11), 6746791 (S100P_CpG_12). Most specifically, at least one CpG site is selected from S100P_CpG_2 at position 6746599, S100P_CpG_3 at position 6746609, S100P_CpG_4 at position 6746616 and S100P_CpG_7 at position 6746710. In particular, the methylation status of at least two, at least three, at least four, at least five, at least six, at least seven, at least eight, at least nine, at least ten, at least eleven, at least twelve, or at least fifteen CpG sites of the present invention is determined. It is understood by the skilled person that the exact numbering of said CpG sites may depend on the specific genomic sequence and on the specific sequence of the S100P promoter region comprised in the sample to be analyzed.

In further embodiments, the method of prognosing and/or diagnosing cancer further comprises the step of comparing the methylation status of the at least one CpG dinucleotide and the presence, in particular the expression level, of the at least one miRNA marker, in said subject, to the methylation status of the at least one CpG dinucleotide and the presence, in particular the expression level, of the at least one miRNA marker in one or more reference(s). In particular, the reference is a threshold value, a reference value or a reference sample.

In embodiments, wherein the reference is a threshold value, a methylation status of the at least on methylation marker selected from the group consisting of HYAL2, MGRN1, RPTOR, SLC22A18, FUT7, RAPSN, and S100P which is below a threshold value is indicative of a subject being afflicted with cancer, an increased risk of developing cancer, or a worsening of the disease; whereas a methylation status which is equal to or above the threshold value is indicative of a subject not afflicted with cancer, of a decreased risk of developing cancer, or of a bettering of the disease. It is to be understood that the aforementioned level may vary due to statistics and errors of measurement.

In embodiments, wherein the reference is a threshold value, an expression level of the at least on methylation marker selected from the group consisting of HYAL2, MGRN1, RPTOR, SLC22A18, FUT7, RAPSN and S100P which is equal to or above the threshold value is indicative of a subject being afflicted with cancer, an increased risk of developing cancer, or a worsening of the disease; whereas an expression level which is below the threshold value is indicative of a subject not being afflicted with cancer, of a decreased risk of developing cancer, or of a bettering of the disease. It is to be understood that the aforementioned level may vary due to statistics and errors of measurement.

In embodiments, wherein the reference is a threshold value, an amount of the at least on miRNA marker selected from the group consisting of miR-148b, miR-409, miR-652, miR-200c, miR-375, miR-320b and miR-141, which is equal to or above the threshold value is indicative of a subject being afflicted with cancer, an increased risk of developing cancer, or a worsening of the disease; whereas an amount which is below the threshold value is indicative of a subject not being afflicted with cancer, of a decreased risk of developing cancer, or of a bettering of the disease. It is to be understood that the aforementioned amounts may vary due to statistics and errors of measurement.

In embodiments, wherein the reference is a reference value, said reference value is a representative value of the absence of cancer, of the presence of cancer, or of an increased or decreased risk of developing cancer.

In further embodiments, the reference sample is selected from the group consisting of a reference sample derived from a healthy individual, a reference sample derived from a diseased individual, a reference sample derived from the same individual as the sample of interest taken at an earlier or later time point, and a reference sample representative for a healthy individual or representative for the presence or absence of cancer or representative for an increased or decreased risk of developing cancer.

In a preferred embodiment of the first aspect of the invention the cancer is breast cancer and/or ovarian cancer. In another preferred embodiment the method is for breast cancer prognosis. In another preferred embodiment the method is for ovarian cancer prognosis. In another preferred embodiment the method is for diagnosing breast cancer. In another preferred embodiment the method is for diagnosing ovarian cancer.

In a preferred embodiment of the first aspect of the invention the methylation status of at least 2, 3, 4, 5, 6, or 7 different CpG dinucleotides is determined. The CpG can be located within the same gene or within different genes.

In a preferred embodiment of the first aspect of the invention the expression level of at least 2, 3, 4, 5, 6, or 7 different miRNAs is determined.

In a preferred embodiment of the first aspect of the invention the methylation status of at least 2, 3, 4, 5, 6, or 7 different CpG dinucleotides is determined and the expression level of at least 2, 3, 4, 5, 6, or 7 different miRNAs is determined.

In a preferred embodiment of the first aspect of the invention the methylation status of at least one CpG dinucleotide within HYAL2 and S100P is determined.

In a preferred embodiment of the first aspect of the invention at least the expression level of miRNAs miR-200c and miR-375 is determined.

In a preferred embodiment of the first aspect of the invention the methylation status of at least one CpG dinucleotide within HYAL2 and S100P is determined and at least the expression level of miRNAs miR-200c and miR-375 is determined.

In a preferred embodiment of the first aspect of the invention the subject has an increased risk of having or developing cancer, preferably breast cancer and/or ovarian cancer.

In a preferred embodiment of the first aspect of the invention the sample is a body fluid sample or a tissue sample, wherein the body fluid sample is preferably selected from the group consisting of blood, serum, plasma, synovial fluid, urine, saliva, lymphatic fluid, lacrimal fluid and fluid obtainable from the glands, and more preferably is peripheral blood.

In a preferred embodiment of the first aspect of the invention the method further comprises the step
(c) determining in vitro the level of cytosine methylation of at least one CpG dinucleotide within and/or expression level of said at least one gene and the expression level of said at least one miRNA in one or more reference samples.

In a preferred embodiment of the first aspect of the invention the CpGs of step a) are selected from HYAL2_CpG1, HYAL2_CpG2, HYAL2_CpG3, HYAL2_CpG4, S100P_CpG_2.3, S100P_CpG4, S100P_CpG7, S100P_CpG8, S100P_CpG9, S100P_CpG10.11.12, SLC22A18_CpG1, SLC22A18_CpG3, SLC22A18_CpG4, SLC22A18_CpG6, SLC22A18_CpG8, RPTOR_CpG1, RPTOR_CpG2, RPTOR_CpG3, RPTOR_CpG5, RPTOR_CpG6, RPTOR_CpG8, RAPSN_CpG1, RAPSN_CpG2, RAPSN_CpG4, RAPSN_CpG5, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, MGRN1_CpG2, MGRN1_CpG4, MGRN1_CpG5.6.7.8, MGRN1_CpG12, MGRN1_CpG15, MGRN1_CpG_16.17.18, MGRN1_CpG19.20, MGRN1_CpG22.23, MGRN1_CpG26.

In a preferred embodiment of the first aspect of the invention in step a) the following CpGs are determined: HYAL2_CpG1, HYAL2_CpG2, HYAL2_CpG3, HYAL2_CpG4, S100P_CpG_2.3, S100P_CpG4, S100P_CpG7, S100P_CpG8, S100P_CpG9, S100P_CpG10.11.12, SLC22A18_CpG1, SLC22A18_CpG3, SLC22A18_CpG4, SLC22A18_CpG6, SLC22A18_CpG8, RPTOR_CpG1, RPTOR_CpG2, RPTOR_CpG3, RPTOR_CpG5, RPTOR_CpG6, RPTOR_CpG8, RAPSN_CpG1, RAPSN_CpG2, RAPSN_CpG4, RAPSN_CpG5, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, MGRN1_CpG2, MGRN1_CpG4, MGRN1_CpG5.6.7.8, MGRN1_CpG12, MGRN1_CpG15, MGRN1_CpG_16.17.18, MGRN1_CpG19.20, MGRN1_CpG22.23, MGRN1_CpG26.

In a preferred embodiment of the first aspect of the invention in step a) the following CpGs are determined: HYAL2_CpG1, HYAL2_CpG2, HYAL2_CpG3, HYAL2_CpG4, S100P_CpG2,3, S100P_CpG7, S100P_CpG8, S100P_CpG9, S100P_CpG10,11,12, SLC22A18_CpG1, SLC22A18_CpG3, SLC22A18_CpG4, SLC22A18_CpG6, RPTOR_CpG1, RPTOR_CpG2, RPTOR_CpG3, RPTOR_CpG5, RPTOR_CpG6, RPTOR_CpG8, RAPSN_CpG1, RAPSN_CpG4, RAPSN_CpG6, RAPSN_CpG7, RAPSN_CpG8, FUT7_CpG1, FUT7_CpG2, FUT7_CpG3, FUT7_CpG4, FUT7_CpG6, FUT7_CpG7, MGRN1_CpG4, MGRN1_CpG5,6,7,8, MGRN1_CpG12, MGRN1_CpG15, MGRN1_CpG16,17,18, MGRN1_CpG19,20, MGRN1_CpG22,23, MGRN1_CpG26. This marker selection is preferably used for breast cancer diagnosis or prognosis, in particular BRCA+ breast cancer diagnosis and prognosis.

In a preferred embodiment of the first aspect of the invention, the expression level of at least miR-200c is determined in step b) and the selection of genes in step a) comprises HYAL2.

In a preferred embodiment of the first aspect of the invention, the expression level of at least miR-200c is determined in step b) and the selection of genes in step a) comprises HYAL2 and S100P.

In a preferred embodiment of the first aspect of the invention, the expression level of at least miR-200c is determined in step b) and the selection of genes in step a) comprises HYAL2, S100P and MGRN1.

In a preferred embodiment of the first aspect of the invention, the expression level of at least miR-375 is determined in step b) and the selection of genes in step a) comprises HYAL2.

In a preferred embodiment of the first aspect of the invention, the expression level of at least miR-375 is determined in step b) and the selection of genes in step a) comprises HYAL2 and S100P.

In a preferred embodiment of the first aspect of the invention, the expression level of at least miR-375 is determined in step b) and the selection of genes in step a) comprises HYAL2, S100P and MGRN1.

In a preferred embodiment of the first aspect of the invention, the expression level of at least miR-320b is determined in step b) and the selection of genes in step a) comprises HYAL2.

In a preferred embodiment of the first aspect of the invention, the expression level of at least miR-320b is determined in step b) and the selection of genes in step a) comprises HYAL2 and S100P.

In a preferred embodiment of the first aspect of the invention, the expression level of at least miR-320b is determined in step b) and the selection of genes in step a) comprises HYAL2, S100P and MGRN1.

In a preferred embodiment of the first aspect of the invention, the expression level of at least miR-200c and miR-320b is determined in step b) and the selection of genes in step a) comprises HYAL2.

In a preferred embodiment of the first aspect of the invention, the expression level of at least miR-200c and miR-320b is determined in step b) and the selection of genes in step a) comprises HYAL2 and S100P.

In a preferred embodiment of the first aspect of the invention, the expression level of at least miR-200c and miR-320b is determined in step b) and the selection of genes in step a) comprises HYAL2, S100P and MGRN1.

In a preferred embodiment of the first aspect of the invention, the expression level of at least miR-200c, miR-320b and miR-375 is determined in step b) and the selection of genes in step a) comprises HYAL2.

In a preferred embodiment of the first aspect of the invention, the expression level of at least miR-200c, miR-320b and miR-375 is determined in step b) and the selection of genes in step a) comprises HYAL2 and S100P.

In a preferred embodiment of the first aspect of the invention, the expression level of at least miR-200c, miR-320b and miR-375 is determined in step b) and the selection of genes in step a) comprises HYAL2, S100P and MGRN1.

In a preferred embodiment of the first aspect of the invention, the method is for diagnosing or prognosing early cancer, preferably early ovarian cancer.

The term “early cancer” as used herein refers to cancer in its early stages. In the art several staging systems are known, which can be used in the present invention to define early or early stage cancer. For example a commonly used staging system for ovarian cancer is the FIGO (International Federation of Gynecology and Obstetrics) system (see www.figo.org). This system uses 3 factors to stage (classify) this cancer: The extent (size) of the tumor (T), the spread to nearby lymph nodes (N) and the spread (metastasis) to distant sites (M). Numbers or letters after T, N, and M provide more details about each of these factors. Higher numbers mean the cancer is more advanced. Once a person's T, N, and M categories have been determined, this information is combined in a process called stage grouping to assign an overall stage.

The staging system in the table below uses the pathologic stage (also called the surgical stage). It is determined by examining tissue removed during an operation. This is also known as surgical staging. Sometimes, if surgery is not possible right away, the cancer will be given a clinical stage instead. This is based on the results of a physical exam, biopsy, and imaging tests done before surgery.

| Stage | | |
|---|---|---|
| grouping | FIGO Stage | Stage description* |
| T1 | I | The cancer is only in the ovary (or ovaries) or fallopian tube(s) (T1). |
| N0 | | It has not spread to nearby lymph nodes (N0) or to distant sites (M0). |
| M0 | | |
| T1a | IA | The cancer is in one ovary, and the tumor is confined to the inside |
| N0 | | of the ovary; or the cancer is in one fallopian tube, and is only inside |
| M0 | | the fallopian tube. There is no cancer on the outer surfaces of the |
| | | ovary or fallopian tube. No cancer cells are found in the fluid |
| | | (ascites) or washings from the abdomen and pelvis (T1a). |
| | | It has not spread to nearby lymph nodes (N0) or to distant sites (M0). |
| T1b | IB | The cancer is in both ovaries or fallopian tubes but not on their |
| N0 | | outer surfaces. No cancer cells are found in the fluid (ascites) or |
| M0 | | washings from the abdomen and pelvis (T1b). It has not spread to |
| | | nearby lymph nodes (N0) or to distant sites (M0). |
| T1c | IC | The cancer is in one or both ovaries or fallopian tubes and any of |
| N0 | | the following are present: |
| M0 | | The tissue (capsule) surrounding the tumor broke |
| | | during surgery, which could allow cancer cells to leak into |
| | | the abdomen and pelvis (called surgical spill). This is stage IC1. |
| | | Cancer is on the outer surface of at least one of the |
| | | ovaries or fallopian tubes or the capsule (tissue surrounding |
| | | the tumor) has ruptured (burst) before surgery (which could |
| | | allow cancer cells to spill into the abdomen and pelvis). |
| | | This is stage IC2. |
| | | Cancer cells are found in the fluid (ascites) or |
| | | washings from the abdomen and pelvis. This is stage IC3. |
| | | It has not spread to nearby lymph nodes (N0) or to distant sites (M0). |
| T2 | II | The cancer is in one or both ovaries or fallopian tubes and has |
| N0 | | spread to other organs (such as the uterus, bladder, the sigmoid |
| M0 | | colon, or the rectum) within the pelvis or there is primary |
| | | peritoneal cancer (T2). It has not spread to nearby lymph nodes |
| | | (N0) or to distant sites (M0). |
| T2a | IIA | The cancer has spread to or has invaded (grown into) the uterus or |
| N0 | | the fallopian tubes, or the ovaries. (T2a). It has not spread to nearby |
| M0 | | lymph nodes (N0) or to distant sites (M0). |
| T2b | IIB | The cancer is on the outer surface of or has grown into other nearby |
| N0 | | pelvic organs such as the bladder, the sigmoid colon, or the rectum |
| M0 | | (T2b). It has not spread to nearby lymph nodes (N0) or to distant |
| | | sites (M0). |
| T1 or T2 | IIIA1 | The cancer is in one or both ovaries or fallopian tubes, or there is |
| N1 | | primary peritoneal cancer (T1) and it may have spread or grown |
| M0 | | into nearby organs in the pelvis (T2). It has spread to the |
| | | retroperitoneal (pelvic and/or para-aortic) lymph nodes only. It has |
| | | not spread to distant sites (M0). |
| | | The cancer is in one or both ovaries or fallopian tubes, or there is |
| | | primary peritoneal cancer and it has spread or grown into organs |
| T3a | IIIA2 | outside the pelvis. During surgery, no cancer is visible in the |
| N0 or N1 | | (outside of the pelvis) to the naked eye, but tiny deposits abdomen |
| M0 | | |
| | | of cancer are found in the lining of the abdomen when it is |
| | | examined in the lab (T3a). |
| | | The cancer might or might not have spread to retroperitoneal lymph |
| | | nodes (N0 or N1), but it has not spread to distant sites (M0). |
| T3b | IIIB | There is cancer in one or both ovaries or fallopian tubes, or there |
| N0 r N1 | | is primary peritoneal cancer and it has spread or grown into organs |
| M0 | | outside the pelvis. The deposits of cancer are large enough for the |
| | | surgeon to see, but are no bigger than 2 cm (about 3/4 inch) across. |
| | | (T3b). |
| | | It may or may not have spread to the retroperitoneal lymph nodes |
| | | (N0 or N1), but it has not spread to the inside of the liver or spleen |
| | | or to distant sites (M0). |
| T3c | IIIC | The cancer is in one or both ovaries or fallopian tubes, or there is |
| N0 or N1 | | primary peritoneal cancer and it has spread or grown into organs |
| M0 | | ioutside the pelvis. The deposits of cancer are larger than 2 cm |
| | | (about 3/4 inch) across and may be on the outside (the capsule) of |
| | | the liver or spleen (T3c). |
| | | It may or may not have spread to the retroperitoneal lymph nodes |
| | | (N0 or N1), but it has not spread to the inside of the liver or spleen |
| | | or to distant sites (M0). |
| Any T | IVA | Cancer cells are found in the fluid around the lungs (called a |
| Any N | | malignant pleural effusion) with no other areas of cancer spread |
| M1a | | such as the liver, spleen, intestine, or lymph nodes outside the |
| | | abdomen (M1a). |
| Any T | IVB | The cancer has spread to the inside of the spleen or liver, to lymph |
| Any N | | nodes other than the retroperitoneal lymph nodes, and/or to other |
| M1b | | organs or tissues outside the peritoneal cavity such as the lungs and |
| | | bones (M1b). |

The above table is an example of a cancer staging system that can be used for ovarian cancer. Early ovarian cancer in the context of the present invention would be FIGO stages I and II (i.e. I, IA, IB, IC, II, IIA and IIB).

Other staging systems for different cancer types are well known in the art.

In a preferred embodiment of the first aspect of the invention, for diagnosing or prognosing early cancer, preferably early ovarian cancer, the expression level of miRNAs miR-148b, miR-652, miR-409, miR200c, miR-375 and miR-320b are determined, optionally the clinical marker CA125 is determined.

In a preferred embodiment of the first aspect of the invention, for diagnosing or prognosing cancer, preferably breast cancer, the expression level of miRNAs miR-148b, miR-652, miR-409, miR200c, miR-375 and miR-320b are determined and the cytosine methylation of at least one CpG dinucleotide within each of the genes HYAL2, MGRN1, RPTOR, SLC22A18, FUT7, RAPSN and S100P is determined, optionally the method further comprises the clinical markers age and/or Qubit.

In an alternative first aspect of the invention a method of diagnosing or prognosing cancer in a subject, comprising the steps of determining in vitro in a sample obtained from said subject

a) the expression level of at least three miRNAs selected from the group consisting of miR-148b, miR-409, miR-652, miR-200c, miR-375 and miR-320b and miR-141, with the proviso that the at least three miRNAs comprise at least the miRNAs miR-200c, miR-375 and miR-320b,

b) optionally the cytosine methylation of at least one CpG dinucleotide within at least one gene selected from the group consisting of HYAL2, MGRN1, RPTOR, SLC22A18, FUT7, RAPSN and S100P and/or

wherein the method optionally further comprises determining the expression level of miR-451a,

wherein the method optionally further comprises determining at least one clinical marker, preferably selected from Age of patient, CA125, cT, cN, cM, pT (Surgery), pN (Surgery), pM and Qubit,

wherein an altered expression level of the at least three miRNAs and if determined a decreased level of cytosine methylation of at least one CpG dinucleotide within the at least one gene is indicative of the present and/or future cancer disease state in said subject.

The embodiments of the first aspect indicated above apply also to the alternative first aspect of the invention.

In a second aspect, the present invention provides a method for diagnosing cancer or for screening for cancer, comprising predicting or detecting the cancer according to the first aspect of the invention. Detecting cancer is to be understood as determining the status of an already existing cancer. This would encompass e.g. diagnosing and prognosing. Predicting cancer does not require the cancer to be present already and would include e.g. providing a measure of susceptibility for cancer or likelihood to develop cancer. The method of the first aspect can also be used for predicting cancer.

In a third aspect, the present invention provides a method for monitoring a subject having an increased risk of developing cancer, comprising predicting or detecting the cancer according to the first aspect of the invention repeatedly.

In a fourth aspect, the present invention provides a method for monitoring cancer treatment of a subject, comprising predicting or detecting the cancer according to the first aspect of the invention repeatedly across the treatment period.

In a fifth aspect, the present invention provides a method for assessing the response of a subject to a cancer treatment, comprising predicting or detecting the cancer according to the first aspect of the invention during and/or after the treatment.

In a sixth aspect, the present invention provides a method for treating a subject having cancer detected according to the method according to the first aspect of the invention, further comprising administering a cancer therapy to the subject.

In a seventh aspect, the present invention provides a kit comprising oligonucleotides for specifically detecting:
the level of cytosine methylation of at least one CpG dinucleotide within from the group consisting of HYAL2, MGRN1, RPTOR, SLC22A18, FUT7, RAPSN and S100P, and/or
the expression level of at least one miRNA selected from the group consisting of 148b, miR-409-3p, miR-652-3p, miR-200c-3p, miR-375, miR-320b, miR-451a and miR-141 with the proviso that the at least one miRNA comprises at least one miRNA selected from the group consisting of miR-200c-3p, miR-375, miR-320b and miR-451a.

In a preferred embodiment the kit is comprising oligonucleotides for specifically detecting:
the level of cytosine methylation of at least one CpG dinucleotide within the genes of the group consisting of HYAL2, MGRN1, RPTOR, SLC22A18, FUT7, RAPSN and S100P, and/or
the expression level of the miRNA from the group consisting of 148b, miR-409-3p, miR-652-3p, miR-200c-3p, miR-375, miR-320b, miR-451a and miR-141.

In preferred embodiments, the kit further comprises

(a) a container, and/or

(b) a data carrier, wherein the data carrier comprises information such as
(i) instructions concerning methods for identifying the risk for developing and/or identifying the presence and/or monitoring progression of cancer
(ii) instructions for use of the means for detecting the methylation status and/or expression level of at least one methylation marker and the amount of at least one miRNA, in particular in a sample, more specifically in a sample from an individual and/or of the kit,
(iii) quality information such as information about the lot/batch number of the means for detecting the methylation status and/or expression level of at least one methylation marker and the amount of at least one miRNA marker and/or of the kit, the manufacturing or assembly site or the expiry or sell-by date, information concerning the correct storage or handling of the kit,
(iv) information concerning the composition of the buffer(s), diluent(s), reagent(s) for detecting the methylation status and/or expression level of at least one methylation marker and the amount of at least one miRNA marker and/or of the means for detecting the methylation status and/or expression level of at least one methylation marker and the amount of at least one miRNA marker,
(v) information concerning the interpretation of information obtained when performing the above-mentioned methods identifying and/or monitoring progression of cancer,
(vi) a warning concerning possible misinterpretations or wrong results when applying unsuitable methods and/or unsuitable means, and/or
(vii) a warning concerning possible misinterpretations or wrong results when using unsuitable reagent(s) and/or buffer(s).

In preferred embodiments, the kit is for use in the method of specified in detail above. In particular, the kid is for use in a method selected from the group consisting of:

(i) a method of diagnosing and/or prognosing cancer, in particular breast cancer and/or ovarian cancer, in a subject, comprising (a) determining the methylation status and/or expression level of at least one methylation marker selected from the group consisting of HYAL2, MGRN1, RPTOR, SLC22A18, FUT7, RAPSN, S100P, and (b) determining the expression level of at least one miRNA marker selected from the group consisting of miR-148b, miR-409, miR-652, miR-200c, miR-375, miR-320b and miR-141, in a subject, wherein the methylation status and/or expression level of at least one methylation marker and the presence of at least one miRNA is indicative of the prognosis and/or diagnosis of said subject,

(ii) a method for determining the dosage of a pharmaceutical for the alteration of cancer or the prevention or treatment of cancer in a subject, comprising the steps of (a) determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b and miR-141, as specified in detail above, in a sample of a subject, and optionally determining the methylation status and/or expression level of at least one methylation marker and the amount of at least one miRNA marker in a reference for comparison with the methylation status and/or expression level of at least one methylation marker and the amount of at least one miRNA marker in the sample of interest, and (b) determining the dosage of a pharmaceutical depending on the methylation status and/or expression level of at least one methylation marker and the amount of at least one miRNA marker in the sample of interest, optionally depending on the comparison of the methylation status and/or expression level of at least one methylation marker and the amount of at least one miRNA marker a in the sample of interest and the reference or reference sample,

(iii) a method for adapting the dosage of a pharmaceutical for the alteration of cancer or the prevention or treatment of cancer, comprising the steps of (a) determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b and miR-141, as specified in detail above, in a sample, (b) determining the methylation status and/or expression level of at least one methylation marker and the amount of at least one miRNA marker in one or more references or reference samples, (c) examining the tested sample as to whether the methylation status and/or expression level of at least one methylation marker and the amount of at least one miRNA marker present in said sample of interest is different from the level in the one or more references or reference samples, and (d) adapting the dosage of a pharmaceutical depending on whether the methylation status and/or expression level of at least one methylation marker and the amount of at least one miRNA marker in the sample of interest is different from the level in the one or more references or reference samples,

(iv) a method of determining the beneficial and/or adverse effects of a substance on cancer or the development of cancer, comprising the steps of (a) determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b, and miR-141, as specified in detail above, in a sample of interest, (b) determining the methylation status and/or expression level of at least one methylation marker and the amount of at least one miRNA marker in one or more references or reference samples, and (c) examining the sample of interest as to whether the methylation status and/or expression level of at least one methylation marker and the amount of at least one miRNA marker present in said sample of interest is different from the level in the one or more references or reference samples, wherein the sample of interest was exposed differently to said substance than the one or more references or reference samples,

(v) a method for identifying a patient as a responder to a cancer treatment, comprising determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b, and miR-141 and, as specified in detail above, in a first sample and in one or more further samples taken subsequently to the first sample, wherein an increased methylation status of the at least one methylation marker and/or a lower expression level of the at least one methylation marker, and the absence or decreased amount of the at least one miRNA marker indicates a response to the treatment,

(vi) a method for identifying a patient as a non-responder to a cancer treatment, comprising determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b, and miR-141, as specified in detail above, in a first sample and in one or more further samples taken subsequently to the first sample, wherein a decreased methylation status of the at least one methylation marker and/or an increased expression level of the at least one methylation marker, and the presence or increased amount of the at least one miRNA marker indicates a lack of response to the treatment, and

(vii) a method for treating cancer, comprising the steps: (i) determining the methylation status and/or expression level of at least one methylation marker selected from the group consisting of HYAL2, MGRN1, RPTOR, SLC22A18, FUT7, RAPSN, S100P, as specified in detail above, and the amount of at least one miRNA marker selected from the group consisting of miR-148b, miR-409, miR-652, miR-200c, miR-375, miR-320b and miR-141, as specified in detail above, in a first sample of a subject; (ii) starting treatment of said patient with a first treatment regimen comprising one or more anti-cancer agents or therapies, (iii) determining the methylation status of at least one methylation marker and/or the expression level of at least one methylation marker, and the amount of at least one miRNA in one or more subsequently taken further samples of said subject; (iv) optionally repeating steps (ii) and (iii) one or more times; (v) continuing treating the patient with the first treatment regimen if there is a substantial increase of the methylation status of the at least one methylation marker and/or a lower expression level of the at least one methylation marker, and a decreased amount or absence of the at least one miRNA marker, or (vi) amending the treatment or terminating treating the patient with the first treatment regimen and treating the patient instead with a second treatment regimen comprising one or more anti-cancer agents or therapies not comprised in the first treatment regimen if there is a decreased methylation status of the at least one methylation marker and/or an increased expression level of the at least one methylation marker, and an increased amount or presence of the at least one miRNA marker.

In an eighth aspect, the present invention provides the use of the kit of the seventh aspect of the invention for predicting, prognosing and/or diagnosing cancer, preferably breast cancer and ovarian cancer.

The following Examples shall merely illustrate the invention. They shall not be construed, whatsoever, to limit the scope of the invention.

### Example 1: Methylation Analysis Using Matrix-Assisted Laser Desorption Ionization Time-of-Flight Mass Spectrometry (MALDI-TOF MS)

A high-throughput quantitative analysis of DNA methylation is performed by MassARRAY assay (Agena BioScience, Inc., Germany), which utilizes base-specific cleavage and MALDI-TOF MS and has been described by Ehrich et al. (Ehrich M. et al., Nucleic Acid Res, 2005. 33(4):pe38). 500 ng of genomic DNA isolated from 200 μl whole blood are used for bisulfite conversion using the EZ DNA methylation-Gold™ kit (Zymo Research, Freiburg, Germany). 1 μl of bisulfite-treated DNA is used as template for amplification by touch-down PCR using bisulfite-specific primers (Table 1). The program used for touch-down PCR is shown in Table 2. After quality control of the PCR products by gel electrophoresis, they are treated according to the standard protocol of the MassARRAY EpiTYPER assay (Agena BioScience Inc., Hamburg, Germany). Briefly, the PCR products are dephosphorylated by Shrimp Alkaline Phosphatase (SAP), in vitro transcribed and cleaved by RNase A. The cleaved products are diluted with ddH₂O up to a final volume of 27 μl. Afterwards 6 mg of CLEAN Resin (Agena BioScience Inc., Hamburg, Germany) is added to the samples to prepare the phosphate backbone of the nucleic acid fragments for the mass spectrometry analysis. The 384-well plate is then centrifuged at 2000 rpm for 2 min and rotated for 30 min.

To carry out the MALDI-TOF MS, 22 nanoliters of each cleavage are robotically dispensed onto a 384-well format SpectroCHIP (Agena BioScience Inc., Hamburg, Germany) prespotted with a matrix of 3-hydroxy-picolinic acid using a Spectropoint nanodispenser. The chips are read by a mass spectrometer (Agena BioScience Inc., Hamburg, Germany). Data is collected by SpectroACQUIRE v3.3.1.3 software and visualized with MassARRAY EpiTYPER v1.0 software. This software automatically provides quantitative results for multiple or single CpG sites across different target sequences.

TABLE 1
Bisulfite-specific primers

| Amplicons | Primers | PrimerID | Sequences |
|---|---|---|---|
| S100P | sense | S100P_F | aggaagagagGGAAGGTGGGTTTGAATTTAGTATT |
| | | | (SEQ ID NO: 16) |
| | antisense | S100P_R | cagtaatacgactcactatagggagaaggctCTATCCCTCTTACCTCTA |
| | | | AACCCCT (SEQ ID NO: 17) |
| SLC22A18 | sense | SLC22A18_F | aggaagagagTAAGTGGAATTTTGGTATTTTTGGA (SEQ |
| | | | ID NO: 18) |
| | antisense | SLC22A18_R | cagtaatacgactcactatagggagaaggctCACTCCAAACCTAAACT |
| | | | CACCTCTA (SEQ ID NO: 19) |
| FUT7 | sense | FUT7_F | aggaagagagGAAGAGGAAGGGATTTAGTTTGAAG (SEQ |
| | | | ID NO: 20) |
| | antisense | FUT7_R | cagtaatacgactcactatagggagaaggctACAAACCTTAACCTCCC |
| | | | AAAATACT (SEQ ID NO: 21) |
| RPTOR | sense | RPTOR_F | aggaagagagGTGGGGTTTTTGTAGTAGTTGAGA (SEQ ID |
| | | | NO: 22) |
| | antisense | RPTOR_R | cagtaatacgactcactatagggagaaggctTAATAACCCAAAACCAA |
| | | | ACCCTAAC (SEQ ID NO: 23) |
| MGRN1 | sense | MGRN1_F | aggaagagagTTTTGGGGTATAAGGGAAGTTTAAG (SEQ |
| | | | ID NO: 24) |
| | antisense | MGRN1_R | cagtaatacgactcactatagggagaaggctCCTAACCAACAAAAAA |
| | | | CCTAAAAAA (SEQ ID NO: 25) |
| RAPSN | sense | RAPSN_F | aggaagagagGATTTTTAGTTGGTGAGAGGTTTGA (SEQ |
| | | | ID NO: 26) |
| | antisense | RAPSN_R | cagtaatacgactcactatagggagaaggctAAAACCACTAAATTACC |
| | | | CAACCAAA (SEQ ID NO: 27) |
| HYAL2 | sense | HYAL2_F | aggaagagagTTTTAAATTTAGTAGGGTGTGAGAGGA |
| | | | (SEQ ID NO: 28) |
| | antisense | HYAL2_R | cagtaatacgactcactatagggagaaggctCTCATCCATATTATAAA |
| | | | AAACCCCC (SEQ ID NO: 29) |

TABLE 2
Program for Touch down PCR
95° C., 5 min
94° C., 30 sec
59° C., 30 sec
72° C., 1 min
go to 2, 4×
94° C., 30 sec
57° C., 30 sec
72° C., 1 min
go to 2, 4×
94° C., 5 sec
55° C., 30 sec
72° C., 1 min
go to 2, 4×
94° C., 5 sec
53° C., 30 sec
72° C., 1 min
go to 2, 35×
72° C., 5 min
4° C., for ever

### Example 2: miRNA Analysis Using Quantitative PCR (qPCR)

Blood Processing and miRNA Isolation from Plasma

EDTA blood samples were collected from cases and control individuals and processed for plasma on the same day. The EDTA tubes were first centrifuged at 1300 g for 20 minutes at room temperature (RT). The supernatant (plasma) was transferred into 2 ml microcentrifuge tubes followed by a second high-speed centrifugation step at 12,000 g for 10 mins (RT) to remove cell debris and fragments. The plasma was aliquoted into cryo vials and stored at −80° C. Circulating miRNAs were extracted from 300 μl plasma using the NucleoSpin miRNA plasma kit (Machery Nagel, Germany) according to the manufacturer's protocol. miRNAs were eluted in 30 μl RNase-free water. miRNA concentration was measured by Qubit Fluorometer (ThermoFischer Scientific, Germany).

Validation of Selected Marker Candidates

Reverse transcription was performed using the MIRCURY LNA kit (Qiagen, Germany) in a final volume of 10 μl. Each reaction comprised of 2 μl RT buffer, 1 μl RT enzyme mix, 2 μl miRNA template and 5 μl RNase-free water. RT was carried out at 42° C. for 1 hour, followed by 5 mins at 95° C. as recommended by the manufacturer. The resulting cDNA was stored at −20° C. and diluted 1:30 directly before use.

Real-time qPCR reactions were performed in duplicate and reactions comprising 2.5 μl PrimaQuant CYBR Mastermix (Steinbrenner, Germany), 0.5 μl specific miRCURY LNA PCR assay (Qiagen, Germany), 0.4 μl nuclease-free water and 1.6 μl diluted cDNA. Real-time PCR was carried out in the qTOWER instrument (Analytik Jena, Germany) under the following conditions:

TABLE 3
Real-time qPCR program

| | | |
|---|---|---|
| 2:00 | 95° C. | Initial denaturation |
| 00:10 | 95° C. | 40× |
| 01:00 | 56° C. | |
| continuous | 60-95° C. | Meltcurve |

### Example 3: Statistical Analysis

In a first step relevant variables (i.e. miRNA and methylation CpG-sites) were selected. Therefore, an elastic net method (H. Zou and T. Hastie. Journal of the Royal Statistical Society, Series B, 67:301-3) was used for variable selection (C. De Mol et al.; Journal of Complexity, 25(2):201-230, April 2009). The elastic net method depends on two hypervariables, which were selected by 5 times cross-validation.

In case wherein variables selected by the elastic net method (i.e. miRNA and methylation CpG-sites) cannot be determined reliably they have been replaced by lower ranked variables.

In a second step the classification between ‘case’ and ‘control’ was established. The preferred methods were tree-based methods such as ‘Random forests’ (L. Breiman, Random forests. In Machine Learning, pages 5-32, 2001). The underlying hypervariables were selected by 5 times cross-validation.

In a third step the models were evaluated. In order to avoid ‘overfitting’ all parameters (i.e. sensitivity, specificity, AUC and accuracy) were determined by 5 times cross-validation.

All statistical analysis was performed with the ‘R’ software package (R Core Team (2018). R: A language and environment for statistical computing. R Foundation for Statistical Computing, Vienna, Austria. URL https://www.R-project.org/). Some analysis additionally included the use of Python 3.

### Example 4: Ovarian Cancer (OC) Marker Panel

The OC marker panel (“OC kit”) is based on the expression levels of circulating miR-148b, -652, -409, -200c, -375 and -320b and can be used for the detection of ovarian cancer with an AUC of 0.89 (Table 4: control vs case).

For early stage ovarian cancer, CA-125 (current gold standard) obtained an AUC of 0.916 alone. In combination with the OC Kit markers, an AUC of 0.94 was reached. When OC is diagnosed in early stages, survival is significantly increased (Table 4: control vs early).

The results are calculated using a stringent machine learning method using two different algorithms (Lasso regression and boosted trees (“Xgboost”)) and comparing their best performance for the defined cohort. Data are randomly split into testing and training sets and further validated by performing a 10-times repeated 5-fold cross procedure to reach reliable results. The obtained overall ROC curve corresponds to the merge of all 10 cross-validations (FIG. 29). In this analysis, the Lasso regression outperformed the Xgboost algorithm and therefore final results provided in table 4 are based on this algorithm. In addition, the particular combination of variables used in the model can have an impact on final output. All statistical analyses have been carried out by using Python (version 3.6.8)

TABLE 4
AUCs, sensitivity, specificity, positive predictive and negative
predictive values are presented for the six microRNA
OC kit markers, the kit markers in combination with
CA-125 and CA-125 alone. AUC values were calculated
based on a 90% specificity.

| GROUP | Model | AUC | Sens | Spec | PPV | NPV |
|---|---|---|---|---|---|---|
| Control vs | Kit | 0.892 | 0.74 | 0.90 | 0.93 | 0.66 |
| case | Kit_CA125 | 0.959 | 0.90 | 0.90 | 0.94 | 0.84 |
| (n = 57 vs 100) | CA125 | 0.960 | 0.87 | 0.90 | 0.94 | 0.80 |
| Control vs. | Kit | 0.818 | 0.69 | 0.90 | 0.79 | 0.84 |
| early | Kit_CA125 | 0.942 | 0.84 | 0.90 | 0.82 | 0.91 |
| (n = 57 vs 31) | CA125 | 0.916 | 0.82 | 0.90 | 0.82 | 0.90 |
| control vs. | Kit | 0.925 | 0.82 | 0.90 | 0.91 | 0.80 |
| late | Kit_CA125 | 0.984 | 0.95 | 0.90 | 0.92 | 0.94 |
| (n = 57 vs 68) | CA125 | 0.982 | 0.91 | 0.90 | 0.92 | 0.90 |

### Example 5: Statistical Methods (Lasso Regression and Xgboost)

1. Data Handling

All analyses have been carried out by using Python, version 3.6.8. Main packages are numpy-1.17.2, pandas-0.25.1, matplotlib-3.1.2, scikit-learn-0.22, xgboost-0.90. Defined features like miRNA expression, methylation levels and/or epidemiological features (i.e. number of pregnancies, age at diagnosis, BMI, etc.) have been previously defined for each analysis. Missing values of features (NA) were imputed (after explorative analysis and before predictive modeling) with random imputation in order to keep them completely uninformative. Random seeds are consistently used throughout the analysis to make every result reproducible.

For the Ovarian cohort, data was additionally standardized (“Z-Score”) as needed by the LASSO algorithm (see section 3 below).

2. Model Selection

Two algorithms were compared regarding the best predictive performance: Lasso regression and boosted trees (“Xgboost”). Comparison was done using a 10-times repeated, stratified 5-fold cross-validation manner (merging prediction of the 5 folds together). For the breast cancer cohort, Xgboost consistently outperformed Lasso regression except for one subgroup (due to low patient count in this subgroup). Therefore, for the breast cancer cohort, all final analysis regarding predictive performance and feature importance are based on Xgboost.

On the other hand, for the ovarian cohort, Lasso regression consistently outperformed or was at least as good as Xgboost but usually had less variance in predictive performance. This might be due to low record count for most cohorts. Therefore, all final analysis regarding predictive performance and feature importance for the ovarian cohort are based on LASSO, using a fixed value of penalty tuning parameter.

3. Predictive Performance

Predictive performance is evaluated by a 10-times repeated 5-fold cross validated procedure with the predictions of all folds merged together per run. So, 10 AUC values are derived and used to estimate the standard deviation (sd; see FIGS. 29 and 30). To get an overall ROC curve, predictions of all 10 runs are also merged.

As Sensitivity, Specificity, PPV (positive predictive value) and NPV (negative predictive value) depend on the cutoff (above which one predicts disease for a patient) as well as on the prevalence, all these metrics are calculated based on a rescaled disease prevalence of 0.011 for breast cancer (applying Bayesian logic) for different cutoffs. Additionally, these metrics are calculated based on a fixed specificity of 0.9.

### Example 6: Breast Cancer (BC) Marker Panel

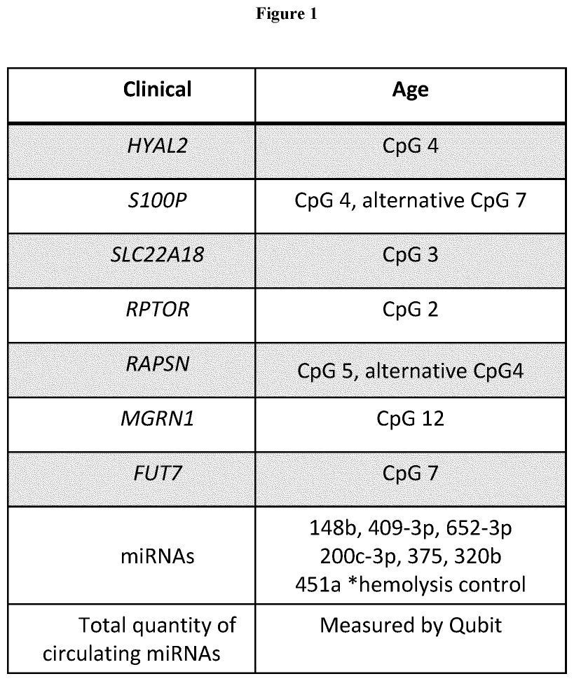

Two marker panels were tested for the diagnosis of breast cancer. The “15 marker BC kit” consists of a combination of Age, the total amount of circulating miRNAs measured by Qubit, the expression level of six circulating microRNA markers consisting of miR-148b, -652,-409, -200c, -375 and -320b and the cytosine methylation in blood of 7 CpG dinucleotides within the following genes HYAL2, MGRN1, RPTOR, SLC22A18, FUT7, RAPSN and S100P (i.e. HYAL2 CpG4, S100P CpG7, SLC22A18 CpG3, RPTOR CpG2, RAPSN CpG4, FUT7 CpG7, MGRN1 CpG12; see FIG. 1). The “14 marker BC kit” excludes age as a marker but is otherwise identical to the “15 marker BC kit”.

In a breast cancer cohort consisting of 216 first diagnosed breast cancer cases compared to 242 healthy controls, the 15 marker BC Kit had an AUC of 0.8 for BC detection (Table 5).

Three additional subgroups were tested (Age as women younger or older than 50 years old, women that have or have not been pregnant and a body mass index (BMI) more or less than 24) as shown in Table 2. Only the subgroup <1 pregnancies showed a better outcome. AUCs were calculated, as for ovarian cancer above, with machine learning algorithms using test and training cohorts and a 10-times repeated 5-fold cross procedure. The XGboost algorithm outperformed the Lasso model in this analysis, thus all results are based on this algorithm.

TABLE 5
AUCs, sensitivity, specificity, positive predictive and negative
predictive values are presented for the combination of six
microRNA markers, amount of circulating miRNAs measured
by Qubit, and seven methylation sites including Age as a variable
(15 marker BC kit) or excluding age (14 maker BC kit)
AUC values were calculated based on a 90% specificity

| Model | Subgroup | AUC | Sens | Spec | PPV | NPV |
|---|---|---|---|---|---|---|
| 15 marker | all | 0.8 | 0.504 | 0.9 | 0.817 | 0.672 |
| BC kit | Age_<50 | 0.708 | 0.175 | 0.9 | 0.421 | 0.725 |
| | Age_>50 | 0.752 | 0.338 | 0.9 | 0.846 | 0.456 |
| | Pregnancies_<1 | 0.87 | 0.571 | 0.9 | 0.724 | 0.82 |
| | Pregnancies_>1 | 0.757 | 0.501 | 0.9 | 0.843 | 0.627 |
| | BMI_<24 | 0.771 | 0.313 | 0.9 | 0.711 | 0.624 |
| | BMI_>24 | 0.768 | 0.502 | 0.9 | 0.833 | 0.646 |
| 14 marker | all | 0.644 | 0.207 | 0.9 | 0.646 | 0.562 |
| BC kit | Age_<50 | 0.594 | 0.116 | 0.9 | 0.326 | 0.711 |
| | Age_>50 | 0.663 | 0.165 | 0.9 | 0.728 | 0.399 |
| | Pregnancies_<1 | 0.661 | 0.206 | 0.9 | 0.486 | 0.712 |
| | Pregnancies_>1 | 0.638 | 0.171 | 0.9 | 0.647 | 0.503 |
| | BMI_<24 | 0.592 | 0.158 | 0.9 | 0.555 | 0.576 |
| | BMI_>24 | 0.606 | 0.169 | 0.9 | 0.626 | 0.522 |
| 15 marker BC | all | 0.772 | 0.425 | 0.9 | 0.789 | 0.639 |
| kit (Lasso | | | | | | |
| logistic | | | | | | |
| regression) | | | | | | |

The performance of different panel of markers was compared to the 15-marker panel in terms of AUC, sensitivity and specificity in order to identify a core marker panel (see Table 6). The Xgboost model was used, as before, as the method of analysis that best suits for this purpose.

TABLE 6
Comparison of the performance of different marker sets

| Marker set | AUC | Sens | Spec | PPV | NPV |
|---|---|---|---|---|---|
| 15 marker BC kit | 0.8 | 0.504 | 0.9 | 0.817 | 0.672 |
| miR-200c, miR-375, miR-320b + Age | 0.753 | 0.438 | 0.9 | 0.795 | 0.645 |
| miR-200c, miR-375, miR-320b, HYAL2 CpG4, | 0.773 | 0.39 | 0.9 | 0.775 | 0.625 |
| S100P CpG7, SLC22A18 CpG3, RPTOR CpG2, | | | | | |
| RAPSN CpG4, FUT7 CpG7, MGRN1 CpG12 + Age | | | | | |
| miR-200c, miR-375, miR-320b, miR-652, | 0.774 | 0.38 | 0.9 | 0.774 | 0.62 |
| HYAL2 CpG4, S100P CpG7, SLC22A18 CpG3, | | | | | |
| RPTOR CpG2, RAPSN CpG4, FUT7 CpG7, | | | | | |
| MGRN1 CpG12 + Age | | | | | |

The best AUC was obtained with the 15-marker panel, however a core marker panel consisting of miR-200c, miR-375 and miR-320b was identified. This panel had only a slightly lower AUC as compared to the full 15-marker panel. The core marker panel with the above mentioned three miRNAs, therefore seems to provide a core panel with good predictive quality that can be further improved by the addition of other markers.

### Items

1. A method of diagnosing or prognosing cancer in a subject, comprising the steps of determining in vitro in a sample obtained from said subject
   a) the expression level of at least three miRNAs selected from the group consisting of miR-148b, miR-409, miR-652, miR-200c, miR-375 and miR-320b and miR-141, with the proviso that the at least three miRNAs comprises at least the miRNAs miR-200c, miR-375 and miR-320b,
   b) optionally the cytosine methylation of at least one CpG dinucleotide within each of at least one gene selected from the group consisting of HYAL2, MGRN1, RPTOR, SLC22A18, FUT7, RAPSN and S100P and/or wherein the method optionally further comprises determining the expression level of miR-451a,
   wherein the method optionally further comprises determining at least one clinical marker, preferably selected from Age of patient, CA125, cT, cN, cM, pT (Surgery), pN (Surgery), pM and Qubit,
   wherein an altered expression level of the at least three miRNAs and if determined a decreased level of cytosine methylation of at least one CpG dinucleotide within the at least one gene is indicative of the present and/or future cancer disease state in said subject.
2. The method of item 1, wherein the following is indicative of the presence of cancer and/or increased likelihood of developing cancer:
   a decreased level of cytosine methylation of at least one CpG dinucleotide within the at least one gene selected from the group consisting of HYAL2, MGRN1, RPTOR, SLC22A18, FUT7, RAPSN and S100P, and/or
   an increase of the expression level of a miRNA selected from miR-148b, miR-409-3p, miR-652-3p, miR-200c-3p, miR-320b and miR-141, and/or
   a decrease of the expression level of miR-375.
3. The method of item 1 or 2, wherein the cancer is breast cancer and/or ovarian cancer, preferably early ovarian cancer.
4. The method of any one of items 1 to 3, wherein
   i) the methylation status of at least 2, 3, 4, 5, 6, or 7 different CpG dinucleotides is determined, and/or
   ii) the expression level of at least 3, 4, 5, 6, or 7 different miRNAs is determined.
5. The method of any one of claims 1 to 4, wherein the methylation status of at least one CpG dinucleotide within HYAL2 and S100P is determined.
6. The method of any one of items 1 to 5, wherein the subject has an increased risk of having or developing cancer.
7. The method of any one of items 1 to 6, wherein the sample is a body fluid sample or a tissue sample, wherein the body fluid sample is preferably selected from the group consisting of blood, serum, plasma, synovial fluid, urine, saliva, lymphatic fluid, lacrimal fluid and fluid obtainable from the glands, and more preferably is peripheral blood.
8. The method of any one of items 1 to 7, wherein the method further comprises the step
   (c) determining in vitro the level of cytosine methylation of at least one CpG dinucleotide within and/or expression level of said at least one gene and the expression level of said at least three miRNA in one or more reference samples.
9. A method for diagnosing cancer or for screening for cancer, comprising predicting or detecting the cancer according to any one of items 1 to 8.
10. A method for monitoring a subject having an increased risk of developing cancer, comprising predicting or detecting the cancer according to any one of items 1 to 8 repeatedly.
11. A method for monitoring cancer treatment of a subject, comprising predicting or detecting the cancer according to any one of items 1 to 8 repeatedly across the treatment period.
12. A method for assessing the response of a subject to a cancer treatment, comprising predicting or detecting the cancer according to any one of items 1 to 8 during and/or after the treatment.
13. A method for treating a subject having cancer detected according to the method according to any one of items 1 to 8, further comprising administering a cancer therapy to the subject.
14. A kit comprising oligonucleotides for specifically detecting:
   the level of cytosine methylation of at least one CpG dinucleotide within at least one gene selected from the group consisting of HYAL2, MGRN1, RPTOR, SLC22A18, FUT7, RAPSN and S100P, and/or
   the expression level of at least three miRNA selected from the group consisting of 148b, miR-409-3p, miR-652-3p, miR-200c-3p, miR-375, miR-320b, miR-451a and miR-141, with the proviso that the at least three miRNA comprises miRNAs miR-200c-3p, miR-375, miR-320b.
15. Use of the kit of item 15 for predicting, prognosing and/or diagnosing cancer, preferably breast cancer and ovarian cancer.

1. A method of diagnosing or prognosing cancer in a subject,
comprising the steps of determining in vitro in a sample obtained from said subject
a) the expression level of at least three miRNAs selected from the group consisting of miR-148b, miR-409, miR-652, miR-200c, miR-375 and miR-320b and miR-141, with the proviso that the wherein at least three miRNAs comprises at least the miRNAs miR-200c, miR-375 and miR-320b,
b) optionally the cytosine methylation of at least one CpG dinucleotide within each of at least one gene selected from the group consisting of HYAL2, MGRN1, RPTOR, SLC22A18, FUT7, RAPSN and S100P and/or
wherein the method optionally further comprises determining the expression level of miR-451a,
wherein the method optionally further comprises determining at least one clinical marker, preferably selected from Age of patient, CA125, cT, cN, cM, pT (Surgery), pN (Surgery), pM and Qubit, and
wherein an altered expression level of the at least three miRNAs and if determined a decreased level of cytosine methylation of at least one CpG dinucleotide within the at least one gene is indicative of the present and/or future cancer disease state in said subject.

2. The method of claim 1, wherein the following is indicative of the presence of cancer and/or increased likelihood of developing cancer:
i) a decreased level of cytosine methylation of at least one CpG dinucleotide within the at least one gene selected from the group consisting of HYAL2, MGRN1, RPTOR, SLC22A18, FUT7, RAPSN and S100P, and/or
ii) an increase of the expression level of a miRNA selected from miR-148b, miR-409-3p, miR-652-3p, miR-200c-3p, miR-320b and miR-141, and/or
iii) a decrease of the expression level of miR-375.

3. The method of claim 1, wherein the cancer is breast cancer and/or ovarian cancer.

4. The method of claim 1, wherein
i) the methylation status of at least 2, 3, 4, 5, 6, or 7 different CpG dinucleotides is determined, and/or
ii) the expression level of at least 2, 3, 4, 5, 6, or 7 different miRNAs is determined.

5. The method of claim 1, wherein
the methylation status of at least one CpG dinucleotide within HYAL2 and S100P is determined.

6. The method of claim 1, wherein the subject has an increased risk of having or developing cancer.

7. The method of claim 1, wherein the sample is a body fluid sample or a tissue sample.

8. The method of claim 1, wherein the method further comprises the step:
(c) determining in vitro the level of cytosine methylation of at least one CpG dinucleotide within and/or expression level of said at least one gene and the expression level of said at least three miRNA in one or more reference samples.

9. A method for diagnosing cancer or for screening for cancer, comprising predicting or detecting the cancer according to claim 1.

10. A method for monitoring a subject having an increased risk of developing cancer, comprising predicting or detecting the cancer according to claim 1 repeatedly.

11. A method for monitoring cancer treatment of a subject, comprising predicting or detecting the cancer according to claim 1 repeatedly across the treatment period.

12. A method for assessing the response of a subject to a cancer treatment, comprising predicting or detecting the cancer according to claim 1 during and/or after the cancer treatment.

13. A method for treating a subject having cancer detected according to the method according to claim 1, further comprising administering a cancer therapy to the subject.

14. A kit comprising oligonucleotides for specifically detecting:
a) the level of cytosine methylation of at least one CpG dinucleotide within at least one gene selected from the group consisting of HYAL2, MGRN1, RPTOR, SLC22A18, FUT7, RAPSN and S100P, and/or
b) the expression level of at least three miRNAs selected from the group consisting of 148b, miR-409-3p, miR-652-3p, miR-200c-3p, miR-375, miR-320b, miR-451a and miR-141, with the proviso that the at least three miRNAs comprises miRNAs miR-200c-3p, miR-375, miR-320b.

15. Use of the kit of claim 14 for predicting, prognosing and/or diagnosing cancer.

16. The method of claim 15, wherein the cancer is breast cancer and/or ovarian cancer.

17. The method of claim 3, wherein the ovarian cancer is early ovarian cancer.

18. The method of claim 7, wherein the body fluid sample is blood, serum, plasma, synovial fluid, urine, saliva, lymphatic fluid, lacrimal fluid and fluid obtainable from the glands.

19. The method of claim 18, wherein the body fluid sample is peripheral blood.


SEQUENCE LISTING (29 sequences)

SEQ ID NO: 1 - RNA, 21 nt, Homo sapiens
aauggcgccacuaggguugug

SEQ ID NO: 2 - RNA, 25 nt, Homo sapiens
caacccuaggagagggugccauuca

SEQ ID NO: 3 - RNA, 22 nt, Homo sapiens
gaauguugcucggugaaccccu

SEQ ID NO: 4 - RNA, 23 nt, Homo sapiens
agguuacccgagcaacuuugcau

SEQ ID NO: 5 - RNA, 22 nt, Homo sapiens
ucagugcaucacagaacuuugu

SEQ ID NO: 6 - RNA, 22 nt, Homo sapiens
aaguucuguuauacacucaggc

SEQ ID NO: 7 - RNA, 23 nt, Homo sapiens
uaauacugccggguaaugaugga

SEQ ID NO: 8 - RNA, 22 nt, Homo sapiens
cgucuuacccagcaguguuugg

SEQ ID NO: 9 - RNA, 22 nt, Homo sapiens
uuuguucguucggcucgcguga

SEQ ID NO: 10 - RNA, 23 nt, Homo sapiens
gcgacgagccccucgcacaaacc

SEQ ID NO: 11 - RNA, 22 nt, Homo sapiens
uaacacugucugguaaagaugg

SEQ ID NO: 12 - RNA, 22 nt, Homo sapiens
caucuuccaguacaguguugga

SEQ ID NO: 13 - RNA, 22 nt, Homo sapiens
aaaccguuaccauuacugaguu

SEQ ID NO: 14 - RNA, 72 nt, Homo sapiens
auaaauuaaucccucucuuucuaguucuuccuagagugaggaaaagcuggguugagagggcaaacaaauuaa

SEQ ID NO: 15 - RNA, 71 nt, Homo sapiens
gucucuuaggcuuucucuucccagauuucccaaaguugggaaaagcuggguugagagggcaaaaggaaaaa

SEQ ID NO: 16 - DNA, 35 bp, Artificial Sequence
  Bisulfite-specific primer S100P_F
aggaagagagggaaggtgggtttgaatttagtatt

SEQ ID NO: 17 - DNA, 56 bp, Artificial Sequence
  Bisulfite-specific primer S100P_R
cagtaatacgactcactatagggagaaggctctatccctcttacctctaaacccct

SEQ ID NO: 18 - DNA, 35 bp, Artificial Sequence
  Bisulfite-specific primer SLC22A18_F
aggaagagagtaagtggaattttggtatttttgga

SEQ ID NO: 19 - DNA, 56 bp, Artificial Sequence
  Bisulfite-specific primer SLC22A18_R
cagtaatacgactcactatagggagaaggctcactccaaacctaaactcacctcta

SEQ ID NO: 20 - DNA, 35 bp, Artificial Sequence
  Bisulfite-specific primer FUT7_F
aggaagagaggaagaggaagggatttagtttgaag

SEQ ID NO: 21 - DNA, 56 bp, Artificial Sequence
  Bisulfite-specific primer
cagtaatacgactcactatagggagaaggctacaaaccttaacctcccaaaatact

SEQ ID NO: 22 - DNA, 34 bp, Artificial Sequence
  Bisulfite-specific primer RPTOR_F
aggaagagaggtggggtttttgtagtagttgaga

SEQ ID NO: 23 - DNA, 56 bp, Artificial Sequence
  Bisulfite-specific primer RPTOR_R
cagtaatacgactcactatagggagaaggcttaataacccaaaaccaaaccctaac

SEQ ID NO: 24 - DNA, 35 bp, Artificial Sequence
  Bisulfite-specific primer MGRN1_F
aggaagagagttttggggtataagggaagtttaag

SEQ ID NO: 25 - DNA, 56 bp, Artificial Sequence
  Bisulfite-specific primer MGRN1_R
cagtaatacgactcactatagggagaaggctcctaaccaacaaaaaacctaaaaaa

SEQ ID NO: 26 - DNA, 35 bp, Artificial Sequence
  Bisulfite-specific primer RAPSN_F
aggaagagaggatttttagttggtgagaggtttga

SEQ ID NO: 27 - DNA, 56 bp, Artificial Sequence
  Bisulfite-specific primer
cagtaatacgactcactatagggagaaggctaaaaccactaaattacccaaccaaa

SEQ ID NO: 28 - DNA, 37 bp, Artificial Sequence
  Bisulfite-specific primer HYAL2_F
aggaagagagttttaaatttagtagggtgtgagagga

SEQ ID NO: 29 - DNA, 56 bp, Artificial Sequence
  Bisulfite-specific primer HYAL2_R
cagtaatacgactcactatagggagaaggctctcatccatattataaaaaaccccc